SEQUENCE LISTING (30 sequences)

SEQ ID NO: 1 - DNA, 1736 bp, Mus musculus
agagctaacgggctggagggaaagaggccgaatgcacacactcctctggccccacttaaggctgccatggggcccagtgctcctctgctgctcctcttctttttgtcatggacgggaccccttcagggacagcagcaccaccttgtggagtacatggaacgccgactagctgccttagaggaacggctggcccaatgccaggatcagagtagtcggcatgctgccgagcttcgggacttcaaaaacaagatgttgcctctcctggaggtggcagagaaggagcgggagaccctcagaactgaagcagactccatctcaggaagagtggaccgtcttgaaagggaggtagactatctggagacacagaacccagctttgccctgtgtagagctggatgagaaggtgactggaggtcctggagccaaaggcaagggccgaagaaatgagaaatacgatatggtgacggactgtagctacacagtcgctcaggtgaggtcaatgaagatcctgaagcggtttggtggttcagttggcctatggaccaaggatccgctggggccagcagagaagatctacgtgttagacggcacccagaacgacacggcttttgtcttcccaaggctgcgtgacttcacccttgccatggctgcccggaaagcttcccgaattcgggtgcccttcccctgggtaggcacggggcagctggtgtacggtggcttcctttattatgctcgaaggcctcctggaggacctggagggggtggtgaattggagaacactctgcagctgatcaaatttcacttggcaaaccgaacagtggtggatagctcagtgttccctgcagagagcctgatacccccctacggcctgacagcagatacatatatcgacctggcagctgatgaggagggcctgtgggctgtctatgccactcgagatgatgacaggcatttgtgtctagccaagttagacccacagacacttgacacagagcagcagtgggacacaccatgtcccagagagaacgcagaggctgcgtttgtcatctgtgggaccctgtacgttgtctataacacccgccctgccagtagggctcgtattcagtgttccttcgatgccagtggtactctcgcccctgaaagggcagcactctcctattttccacgccgatatggtgcccatgccagccttcgctataacccccgtgagcgccagctgtatgcctgggatgatggctaccagattgtctacaaattggagatgaagaagaaggaggaggaagtttaagcagctagccttgtgctcttgattcttatgcccagacatttatattcctgtgagctctcctgcagttcatccttcaaaacgaaggccagtggtggtagctcatataccctaatttctaaaggacaaccaaattctcaagcccctctgttttatgcagaactccagatcctgggtagcattttagaactgaacagcaaacaaacaccctaaatcttcactcctgccttatgtccacaaagtttagttccaaactcagagccctgtcctttggagagggtcaaccccagacagcaggcgacagcattcttgccctcagtatgaccgaagggagagaactcagagacaaagctgccctccctcccttccccctccagtgtaggggagaatggggctttccccacatcactttgtatggtaacagtttgcattaaaaggaaaacccaccattc

SEQ ID NO: 2 - DNA, 1852 bp, Homo sapiens
caggagagaaggcaccgcccccaccccgcctccaaagctaaccctcgggcttgaggggaagaggctgactgtacgttccttctactctggcaccactctccaggctgccatggggcccagcacccctctcctcatcttgttccttttgtcatggtcgggacccctccaaggacagcagcaccaccttgtggagtacatggaacgccgactagctgctttagaggaacggctggcccagtgccaggaccagagtagtcggcatgctgctgagctgcgggacttcaagaacaagatgctgccactgctggaggtggcagagaaggagcgggaggcactcagaactgaggccgacaccatctccgggagagtggatcgtctggagcgggaggtagactatctggagacccagaacccagctctgccctgtgtagagtttgatgagaaggtgactggaggccctgggaccaaaggcaagggaagaaggaatgagaagtacgatatggtgacagactgtggctacacaatctctcaagtgagatcaatgaagattctgaagcgatttggtggcccagctggtctatggaccaaggatccactggggcaaacagagaagatctacgtgttagatgggacacagaatgacacagcctttgtcttcccaaggctgcgtgacttcacccttgccatggctgcccggaaagcttcccgagtccgggtgcccttcccctgggtaggcacagggcagctggtatatggtggctttctttattttgctcggaggcctcctggaagacctggtggaggtggtgagatggagaacactttgcagctaatcaaattccacctggcaaaccgaacagtggtggacagctcagtattcccagcagaggggctgatccccccctacggcttgacagcagacacctacatcgacctggcagctgatgaggaaggtctttgggctgtctatgccacccgggaggatgacaggcacttgtgtctggccaagttagatccacagacactggacacagagcagcagtgggacacaccatgtcccagagagaatgctgaggctgcctttgtcatctgtgggaccctctatgtcgtctataacacccgtcctgccagtcgggcccgcatccagtgctcctttgatgccagcggcaccctgacccctgaacgggcagcactcccttattttccccgcagatatggtgcccatgccagcctccgctataacccccgagaacgccagctctatgcctgggatgatggctaccagattgtctataagctggagatgaggaagaaagaggaggaggtttgaggagctagccttgttttttgcatctttctcactcccatacatttatattatatccccactaaatttcttgttcctcattcttcaaatgtgggccagttgtggctcaaatcctctatatttttagccaatggcaatcaaattctttcagctcctttgtttcatacggaactccagatcctgagtaatccttttagagcccgaagagtcaaaaccctcaatgttccctcctgctctcctgccccatgtcaacaaatttcaggctaaggatgccccagacccagggctctaaccttgtatgcgggcaggcccagggagcaggcagcagtgttcttcccctcagagtgacttggggagggagaaataggaggagacgtccagctctgtcctctcttcctcactcctcccttcagtgtcctgaggaacaggactttctccacattgttttgtattgcaacattttgcattaaaaggaaaatccactgctaaaaaaaaaaaaaaaaaaaaaaaaaaaaaaaaaaaaaa

SEQ ID NO: 3 - DNA, 2276 bp, Rattus norvegicus
gccttacttaaggctgccatggggcccagtgctcctctgctcctcttcttccttttgtcatggccgggaccccttcagggacagcagcaccaccttgtggagtacatggaacgccgactagctgccttagaggtgagggacttgtttctcttctcgaccgcccgggatgattccacgtctataggtgcagctagttgtcagggctctgggagtgacaagatcggactgtgtaagaagtctttaatgacgagtaagcctcctaattacaattatttccccaacataaaacagttgtccagcaaataaatgcaacacatttccctcaggattagatggaataaggggacgggaaatgggagtcagaatcgggctgcatctaagaactgtctggctatattgttcaagcttcccaggcaatctgcctgcaacttcctgtctgtttcttccccgcaccccacatctgctcgcttttgaagagtttgaagtctgtaacatcctaatttccaggagtctcagttcactgagaacacaggctgaggtgaattaagccaggttctttgatagggatggtagtactgactagtctcctttgctgtgggagcatggctgagcttgtccctttcctggttccatctccccggtagggcctttgcttgcctgggagttatagccctgcagcccgccccttctctggtttccccggcccgtcaggagcggctggcacagtgccaggatcagagcagtcggcatgctgctgagcttcgggacttcaaaaacaagatgctgcctctactggaggtggcagagaaggagcgggaaacactcagaaccgaggcagacagcatttcaggaagagtggaccgtcttgaacgggaagtagactacctggagacacagaacccagctttgccctgtgtagaactggatgagaaggtgactggaggccctggaaccaaaggcaagggccggagaaatgagaaatacgatatggtgacagactgtagctacacaatctctcaggtgaggtcaatgaagatcctgaagcggtttggtggctcagctggcctatggaccaaggatccactggggccagcagagaagatctacgtgttagacggcacgcagaacgacacggccttcgtcttcccgaggttgcgtgacttcaccctcaccatggctgcccgcaaagcctcccgaattcgggtgcccttcccctgggtaggcacaggacagctggtgtatggtggcttcctttattatgctcgaaggcctcctggaggagctggagggggtggtgagttggagaatactctgcagctgatcaaatttcacttggcaaaccgaacagtggtggacagctcagtgttccctgcagagagactgatacccccctacgggctgacagtagacacatatatcgacctggcagctgatgaggagggcctttgggctgtctatgccactcgggaagatgacaggcatttgtgtctagccaagttagacccacagacgctcgacacagagcagcagtgggacacgccgtgtcccagagagaatgcagaggctgcgttcgtcatctgtgggaccctgtacgtggtctataacacccgtcctgccagtagagctcgtattcagtgctcctttgatgccagtggtactctcacccctgaaagggcagcactctcctattttccacgccgatatggtgcccatgccagccttcgctataacccccgtgagcgccagctgtatgcctgggacgatggctaccagattgtctacaagctggagatgaagaagaaggaggaggaagtttaagagctagccttgtgcttttgattcttatgcccagacatttatattcctgtgagatctcctgaagatcattcttcaaaacgatggccaatggtgctggctcatttaccctaatttttaaacgacagccaaccaaattctcatagcccctctggtttatgcagaactccagatcttgagtagtttttagaagtaaacagcaaacaccttaaatcttcactcctgctctctcctgctctgtgtccacaaagtttagttccaaactgagagccctgtcctttggttagggtcaaccccagacagcaggcgacagtattcttgccctcagtgtgacccaaggggggaaactcagagacaacactgccctccctccctcagccctccagtgtaggggcgaatggggctttcctcacatcactctgtatggcaacattttgcattaaaaggaaaacccaccactc

SEQ ID NO: 4 - DNA, 1179 bp, Gallus gallus
atggggccctggcgctgtctgctcctcctgccgctcctcgccgcggcaccgcgcgcacagcagcagcagttcatggagtacgtggagcggcggttggcgctgctggaggagaggatcgcgcagtggcacgatcagagcagccgctactccacggagctgcgggacttcaagaaccaggtgctgggcatgctggagacggcggagaaggagcgcgaggcgctgcgggccgaggcggaaggagcggcggcgcgggtggaccgcctggagcgagaggtggactacctggaaacgcagaaccccgcaccgccctgcgtggaggtggacgaagtgctgatggagaagcaggcggccacggccaagcagaggaagaacgagaagtacaccaaactgacggattgcagtgacaccattgcaagtgtccgagccatgaagatcctgaagcgcttcggcagctctgcggggctctggaccaaggatgctgcggggagctccgagaagatctacgtcttcgacggcactgccaacgacacggtgtacatcttcccccgcatgcgggagttcacgctcttctcggccacgcgccgtgcagcacgcatcaagctgccctacccctgggtgggcaccgggcacctggtctatggcgggtacctctactacatccgccagcagggccccttccaggtgatcaagtttgacctggccaacaagacggtggtggacagctcggtgttcccggccgaggagcagatccccgtcttcgggctctcccccttcacctacatcgagctggcggcggatgaagaggggctgtgggccatctacgccaccaaagagaacgagaagaacatctgcctggccaagctggaccccgactcgctggacatcgagcagatgtgggacacgccgtgcccgcgggagaacgccgagggcgccttcgtggtgtgcggggctctgcacgtggcctacaacacgcgcctgcccagccgctcccgcgtgcagtgcgtcttcgacgtcagtgggacgctgccccccgaggaggcctccctcgtgtacttccccaaacgttacggctcccattccagcatgaagtacagcccacgcgagaggcaggtctacgcctgggacgacggctaccagatcatctaccgcatggagatgaagaagaagctggaggtctga

SEQ ID NO: 5 - DNA, 2719 bp, Homo sapiens
aatgcacacacaattaagccaggaagcagcttgcaaccactagcctggggagggtccgcatgtgtcaagggtgagggcaacagatgctggacccagggagctctctgccacaggtcagtctacaaggcctcagggaccaacttgccaacagctggacttgatcactagctggcaaactgagctcacgtatcgggtggaataacaagcggactttgctctctgctgtgcaaaacgctgtttttagaggatttgccacagcagcggatagagcaggagagcaccaccggagcccttgagacatccttgagaagagccacagcataagagactgccctgcttggtgttttgcaggatgatggtggcccttcgaggagcttctgcattgctggttctgttccttgcagcttttctgcccccgccgcagtgtacccaggacccagccatggtgcattacatctaccagcgctttcgagtcttggagcaagggctggaaaaatgtacccaagcaacgagggcatacattcaagaattccaagagttctcaaaaaatatatctgtcatgctgggaagatgtcagacctacacaagtgagtacaagagtgcagtgggtaacttggcactgagagttgaacgtgcccaacgggagattgactacatacaataccttcgagaggctgacgagtgcatcgtatcagaggacaagacactggcagaaatgttgctccaagaagctgaagaagagaaaaagatccggactctgctgaatgcaagctgtgacaacatgctgatgggcataaagtctttgaaaatagtgaagaagatgatggacacacatggctcttggatgaaagatgctgtctataactctccaaaggtgtacttattaattggatccagaaacaacactgtttgggaatttgcaaacatacgggcattcatggaggataacaccaagccagctccccggaagcaaatcctaacactttcctggcagggaacaggccaagtgatctacaaaggttttctattttttcataaccaagcaacttctaatgagataatcaaatataacctgcagaagaggactgtggaagatcgaatgctgctcccaggaggggtaggccgagcattggtttaccagcactccccctcaacttacattgacctggctgtggatgagcatgggctctgggccatccactctgggccaggcacccatagccatttggttctcacaaagattgagccgggcacactgggagtggagcattcatgggataccccatgcagaagccaggatgctgaagcctcattcctcttgtgtggggttctctatgtggtctacagtactgggggccagggccctcatcgcatcacctgcatctatgatccactgggcactatcagtgaggaggacttgcccaacttgttcttccccaagagaccaagaagtcactccatgatccattacaaccccagagataagcagctctatgcctggaatgaaggaaaccagatcatttacaaactccagacaaagagaaagctgcctctgaagtaatgcattacagctgtgagaaagagcactgtggctttggcagctgttctacaggacagtgaggctatagccccttcacaatatagtatccctctaatcacacacaggaagagtgtgtagaagtggaaatacgtatgcctcctttcccaaatgtcactgccttaggtatcttccaagagcttagatgagagcatatcatcaggaaagtttcaacaatgtccattactcccccaaacctcctggctctcaaggatgaccacattctgatacagcctacttcaagccttttgttttactgctccccagcatttactgtaactctgccatcttccctcccacaattagagttgtatgccagcccctaatattcaccactggcttttctctcccctggcctttgctgaagctcttccctctttttcaaatgtctattgatattctcccattttcactgcccaactaaaatactattaatatttctttcttttcttttcttttttttgagacaaggtctcactatgttgcccaggctggtctcaaactccagagctcaagagatcctcctgcctcagcctcctaagtacctgggattacaggcatgtgccaccacacctggcttaaaatactatttcttattgaggtttaacctctatttcccctagccctgtccttccactaagcttggtagatgtaataataaagtgaaaatattaacatttgaatatcgctttccaggtgtggagtgtttgcacatcattgaattctcgtttcacctttgtgaaacatgcacaagtctttacagctgtcattctagagtttaggtgagtaacacaattacaaagtgaaagatacagctagaaaatactacaaatcccatagtttttccattgcccaaggaagcatcaaatacgtatgtttgttcacctactcttatagtcaatgcgttcatcgtttcagcctaaaaataatagtctgtccctttagccagttttcatgtctgcacaagacctttcagtaggcctttcaaatgataattcctccagaaaaccagtctaagggtgaggaccccaactctagcctcctcttgtcttgctgtcctctgtttctctctttctgctttaaattcaataaaagtgacactgagcaaaaaaaaaaaaaaaa

SEQ ID NO: 6 - DNA, 1969 bp, Mus musculus
acactctgaacctggaagctgtaagtagccattagcctggagagggtctgtgcatggccaagctgagggcagcagacactggacccccggagctctctgcaaccaagtgaacacgtaaggcctgcagagactggcttgccaacagctggacttgatcaccagcttgaaactgagatcatgcaccgggtagaataacaagcggactttgttctctctggctatgcaaagcgttgttttccaaggaattgccacagcagcagacagaaaaccagagatcaccacccggacccctgagtcgttttagagaaccagagttcctgggaagtgtgcaggatggtggtggcccttcaagaagcttctgcttccctggttctcttccttgcagcatttctgcccccaccacaacatgcccaggacccagccatggtacactacatctaccagcgcttccaagtcctcgagcaagggctggaaaaatgtgcccaaacaacaagggcatatattcaagacttccaggaattctcaaaaaacatatccatcatgctggggaggtgtcagacccacacaagtgaatataagagtgcagtggaaaaccttgccctaagagtggagcgtgcccagcaggagatcgactacctgcagtacctgcgagaagctgacttctgcattgaatctgaggagaagacactggctgaaaagttgcttcaagaagaagcagaagaaaagaagatccgaaccctgttgaacacaagctgtgacaacatgctgatggctataaagtctctgaaaatagtgaagaagaccgtggacccagatggctcttggatgaaagatgctggcagtaactctgcaaaagtgtatctattagctggatccagaaacaacacagtttgggaatttgcaaacttgcgggcattcatggaagatagcatcaagcctggtccccgtaagttaatcttaccactttcctggcagggatcagggcaagtggtctaccaaagctttctatttttttcacaatcaaggaacttctaatgagataattaaatataatctgcagaagaagactgtggaagatcgaatgcttctcccaggaggggcaggccgagcacccatctatcaacactctctctctacgtacattgaccttgctgtggatgaacacggactctgggccattcactcaggaccaggtatccaaggccatttggttctcacaaaaattgagcctgacactttgggagtagaacattcatgggatacaccgtgtagaagccaggatgcggaagcttcattcctactgtgtggggtcctctatgttgtctacagttccggtggccagggctctcatcgaatcacctgtgtctatgatccactgggcactgtcagggaggagcatctgcccaatttgttcttccctaggcggccaagaagtcactccatgatccattacaaccccagagataagcagctttatgcctggaatgaaggcaatcagatcatttacaaactccagacaaagaaaaagctgcctctggagtaatggctggcattggagggagagcagtgtgacttgggcagtggttttccgggacactgaggtcacagcccctttatagtggagtatctggccctctcgatgcacataggaataggttctaaaagttgaaatacatattcttcctttcccaaatgtcactcactgccttaagtatcttttgaaagcatggatgcggtcacagttcaattgatcaataccctaccctgacccaaatctgatggggcttaaggcctcttttgtctggtttacttacatccaatctttccttttgccttccagcatttcctctaattctgctctattccccatccagcaattaggatcttgccagtcctggtattgacttctctggacttcactcagacgctgtcctctttttcaaatacttataaagattttccaatgtcc

SEQ ID NO: 7 - DNA, 2953 bp, Rattus norvegicus
gtagccactagcctgagagggtctgtgcatgtccaagctgagggcagcagatgctggacctcgggagccctctgcaccaagtgaacaaggcaggcctgcagagaccagcttgccaacagctggacttgatcaccagcttgaagctgagatcatgcactgggtagaataacaagcggactttgttctctctggctatgcaaagtgttgttttccaaggaattgccacagcagcagacagaaaaccagagctcaccacctggacccttgagtcatttcagagaaccaaagttcctgggaagtgtgcaggatgatggtggcccttccgggagcttctgcttcactggttctctttctcgcagcatttctgcccccactacaacatgcccaggacccagccatggtacactacatctaccagcgcttccaagtcttggagcaagggctggaaaaatgtgcccaaacaacaagggcctatattcaagatttccaggaattctcaaaaaacctatccaccatgctggggaggtgtcagacccacacgaatgagtacaggagtgcagtggataaccttgccctgagagtggagcgtgcccagcgggagatcgactacctgcaatacctcagggaatctgacttctgcgttgaatcggaggagaagacatcagctgaaaaggtgcttcaagaagcagaagaagaaaagaagatccgaaccctgttgaacacaagctgtgacaacatgctaatggctataaagtctctgaaaatagtgaagaagaccgtggacccagagggctcttggatgaaggatgctggcagtacctctgctaaagtgtatctattagctggatccagaaacaacacagtttgggaatttgcaaacttgcgggccttcatggaagatagtgtgaagcctggtccccggaagctaaccttaccactttcctggcagggatcagggcaagtggtctaccagagtttcctattttttcacaatcaaggaacttctaatgagataattaaatataatctgcagaagaagactgtggaagatcgaatgcttcttccaggaggggcaggccgagcacccatctaccaacactccctctctacatacattgaccttgctgtggatgaacatggactgtgggccattcactcaggaccaggtatccaaggccatttggttctcacaaaaattgaggctggcactttaggaatagagcattcatgggatacgccgtgtagaagccaggatgctgaagcatcattcctcctgtgtggggtcctctatgttgtctacagttccggtggccagggccctcatcgaatcacctgtgtctatgacccactgggcactgtcagggaggagcatctgcccaatttgttcttccctaggcgggcaaggagtcactccatgatccattacaaccccagagataagcagctgtatgcctggaatgaaggctatcagatcatttacaaactccagacaaagaaaaaactgcctctggagtaatggacagcagtttgcagaaagagttgtgtggcttaggcagtggtttttccgggacactgaggtcatggcccctttacattggagtatcataggaatgagttctaaaatttaaaatacatattctttctttctcaaatgttaccgccttaggtatcttttgagagcatagatgaggtcacaatttagaccaataccttgccctcacccaaatctgatggggattaaggtctcttccatctggtttagttatttccaatccttccttttttctttcagcattttctctgactctgccctatttcttatccagcaactaggatcttgcccgtcctggtgttgacttcttctctggacttcactcaggctctgttctctttttcaaatgcttataaagatttttcattgtccaactgtacatttcttgttgctgacaagaagcaacttaagagagaaagggtttataccagcttattgttttaaaagaagccaggcacagtggtgcacaccttttaccctggtctccaggaagcttaggaaggtggatctctgtgtgtttgaggtcaacctggtctacatggtaagttccagggccaacagggcaatacagtgagcgcttgtcttaaaaaaaaatggatatagtccaccatagtagtagtgaaagtatggtagcaggagcatcagatgttcaagtcacatctgtaatcaggatgcaggaagcaaacaggaagaaaggctacatacactaagaaacttaaaggcttgctctcatagtaacgcactactcgtcatagtgacacaagacctgcattcagctgggaaccaagcgttcatgtgagcttctggggcacatactcatcggaccacaacacccaggaaacactatttcttcttggtgcctaatttttatttcctctagccttgctcttctgtcaagccaagtggatacagtaacagtgtgaaatcatgaatagttggatgttactttctagttacaaagctctttttacattgtttaattgtcttttctctttgtcaggtttgtacacatcttcatagttgctaagttagaggtgagagttcaggtgagttattcaaagtagcaaactacaaaagagctagaaagactgcaaacctcttagtatttccatcatccaagaaaggaaatatcaattaggcacttttcccacttatccttaatcagtgtgtccatcagttcagcctaaaaataacactctgccccttagccagttttcatgtatgcacaacgtcttatatataggtctttcaaagtctgctttatctaaaaaaccagtccaaggtaaggatgcaattctagcctcatcttgtttttttgttgcctgcttctttcttcttgctttcagtgtaataaaagtgagattaagcagataaaaaaaaaaaaaaaaaaaaaaaaaaaaa

SEQ ID NO: 8 - RNA, 25 nt, Artificial Sequence
  Synethic primer
ggcucguauucaguguuccuucgau

SEQ ID NO: 9 - RNA, 25 nt, Artificial Sequence
  Synthetic primer
gauauggugacggacuguagcuaca

SEQ ID NO: 10 - RNA, 25 nt, Artificial Sequence
  Synthetic primer
gccacucgagaugaugacaggcauu

SEQ ID NO: 11 - protein, 406 aa, Mus musculus
MGPSAPLLLLFFLSWTGPLQGQQHHLVEYMERRLAALEERLAQCQDQSSRHAAELRDFKNKMLPLLEVAEKERETLRTEADSISGRVDRLEREVDYLETQNPALPCVELDEKVTGGPGAKGKGRRNEKYDMVTDCSYTVAQVRSMKILKRFGGSVGLWTKDPLGPAEKIYVLDGTQNDTAFVFPRLRDFTLAMAARKASRIRVPFPWVGTGQLVYGGFLYYARRPPGGPGGGGELENTLQLIKFHLANRTVVDSSVFPAESLIPPYGLTADTYIDLAADEEGLWAVYATRDDDRHLCLAKLDPQTLDTEQQWDTPCPRENAEAAFVICGTLYVVYNTRPASRARIQCSFDASGTLAPERAALSYFPRRYGAHASLRYNPRERQLYAWDDGYQIVYKLEMKKKEEEV

SEQ ID NO: 12 - protein, 406 aa, Homo sapiens
MGPSTPLLILFLLSWSGPLQGQQHHLVEYMERRLAALEERLAQCQDQSSRHAAELRDFKNKMLPLLEVAEKEREALRTEADTISGRVDRLEREVDYLETQNPALPCVEFDEKVTGGPGTKGKGRRNEKYDMVTDCGYTISQVRSMKILKRFGGPAGLWTKDPLGQTEKIYVLDGTQNDTAFVFPRLRDFTLAMAARKASRVRVPFPWVGTGQLVYGGFLYFARRPPGRPGGGGEMENTLQLIKFHLANRTVVDSSVFPAEGLIPPYGLTADTYIDLAADEEGLWAVYATREDDRHLCLAKLDPQTLDTEQQWDTPCPRENAEAAFVICGTLYVVYNTRPASRARIQCSFDASGTLTPERAALPYFPRRYGAHASLRYNPRERQLYAWDDGYQIVYKLEMRKKEEEV

SEQ ID NO: 13 - protein, 418 aa, Rattus norvegicus
MHTPTLALLKAAMGPSAPLLLFFLLSWPGPLQGQQHHLVEYMERRLAALEERLAQCQDQSSRHAAELRDFKNKMLPLLEVAEKERETLRTEADSISGRVDRLEREVDYLETQNPALPCVELDEKVTGGPGTKGKGRRNEKYDMVTDCSYTISQVRSMKILKRFGGSAGLWTKDPLGPAEKIYVLDGTQNDTAFVFPRLRDFTLTMAARKASRIRVPFPWVGTGQLVYGGFLYYARRPPGGAGGGGELENTLQLIKFHLANRTVVDSSVFPAERLIPPYGLTVDTYIDLAADEEGLWAVYATREDDRHLCLAKLDPQTLDTEQQWDTPCPRENAEAAFVICGTLYVVYNTRPASRARIQCSFDASGTLTPERAALSYFPRRYGAHASLRYNPRERQLYAWDDGYQIVYKLEMKKKEEEV

SEQ ID NO: 14 - protein, 392 aa, Gallus gallus
MGPWRCLLLLPLLAAAPRAQQQQFMEYVERRLALLEERIAQWHDQSSRYSTELRDFKNQVLGMLETAEKEREALRAEAEGAAARVDRLEREVDYLETQNPAPPCVEVDEVLMEKQAATAKQRKNEKYTKLTDCSDTIASVRAMKILKRFGSSAGLWTKDAAGSSEKIYVFDGTANDTVYIFPRMREFTLFSATRRAARIKLPYPWVGTGHLVYGGYLYYIRQQGPFQVIKFDLANKTVVDSSVFPAEEQIPVFGLSPFTYIELAADEEGLWAIYATKENEKNICLAKLDPDSLDIEQMWDTPCPRENAEGAFVVCGALHVAYNTRLPSRSRVQCVFDVSGTLPPEEASLVYFPKRYGSHSSMKYSPRERQVYAWDDGYQIIYRMEMKKKLEV

SEQ ID NO: 15 - protein, 402 aa, Homo sapiens
MMVALRGASALLVLFLAAFLPPPQCTQDPAMVHYIYQRFRVLEQGLEKCTQATRAYIQEFQEFSKNISVMLGRCQTYTSEYKSAVGNLALRVERAQREIDYIQYLREADECIVSEDKTLAEMLLQEAEEEKKIRTLLNASCDNMLMGIKSLKIVKKMMDTHGSWMKDAVYNSPKVYLLIGSRNNTVWEFANIRAFMEDNTKPAPRKQILTLSWQGTGQVIYKGFLFFHNQATSNEIIKYNLQKRTVEDRMLLPGGVGRALVYQHSPSTYIDLAVDEHGLWAIHSGPGTHSHLVLTKIEPGTLGVEHSWDTPCRSQDAEASFLLCGVLYVVYSTGGQGPHRITCIYDPLGTISEEDLPNLFFPKRPRSHSMIHYNPRDKQLYAWNEGNQIIYKLQTKRKLPLK

SEQ ID NO: 16 - protein, 233 aa, Mus musculus
MVVALQEASASLVLFLAAFLPPPQHAQDPAMVHYIYQRFQVLEQGLEKCAQTTRAYIQDFQEFSKNISIMLGRCQTHTSEYKSAVENLALRVERAQQEIDYLQYLREADFCIESEEKTLAEKLLQEEAEEKKIRTLLNTSCDNMLMAIKSLKIVKKTVDPDGSWMKDAGSNSAKVYLLAGSRNNTVWEFANLRAFMEDSIKPGPRKLILPLSWQGSGQVVYQSFLFFSQSRNF

SEQ ID NO: 17 - protein, 402 aa, Rattus norvegicus
MMVALPGASASLVLFLAAFLPPLQHAQDPAMVHYIYQRFQVLEQGLEKCAQTTRAYIQDFQEFSKNLSTMLGRCQTHTNEYRSAVDNLALRVERAQREIDYLQYLRESDFCVESEEKTSAEKVLQEAEEEKKIRTLLNTSCDNMLMAIKSLKIVKKTVDPEGSWMKDAGSTSAKVYLLAGSRNNTVWEFANLRAFMEDSVKPGPRKLTLPLSWQGSGQVVYQSFLFFHNQGTSNEIIKYNLQKKTVEDRMLLPGGAGRAPIYQHSLSTYIDLAVDEHGLWAIHSGPGIQGHLVLTKIEAGTLGIEHSWDTPCRSQDAEASFLLCGVLYVVYSSGGQGPHRITCVYDPLGTVREEHLPNLFFPRRARSHSMIHYNPRDKQLYAWNEGYQIIYKLQTKKKLPLE

SEQ ID NO: 18 - protein, 10 aa, Artificial Sequence
  Synthetic peptide
RARIQCSFDA

SEQ ID NO: 19 - protein, 9 aa, Artificial Sequence
  Synthetic peptide
DMVTDCSYT

SEQ ID NO: 20 - protein, 9 aa, Artificial Sequence
  Synthetic peptide
DMVTDCGYT

SEQ ID NO: 21 - protein, 9 aa, Artificial Sequence
  Synthetic peptide
ATRDDDRHL

SEQ ID NO: 22 - protein, 9 aa, Artificial Sequence
  Synthetic peptide
ATREDDRHL

SEQ ID NO: 23 - DNA, 22 bp, Artificial Sequence
  Synthetic primer
gctgtctatgccactcgagatg

SEQ ID NO: 24 - DNA, 24 bp, Artificial Sequence
  Synthetic primer
tgtgtcaagtgtctgtgggtctaa

SEQ ID NO: 25 - DNA, 21 bp, Artificial Sequence
  Synthetic primer
atgctcctggctctgggactc

SEQ ID NO: 26 - DNA, 42 bp, Artificial Sequence
  Synthtic primer
ctaatacgactcactatagggtgcagctggtccccttctatg

SEQ ID NO: 27 - DNA, 42 bp, Artificial Sequence
  Synthetic primer
ctaatacgactcactatagggagtgctcctctgctgctcctc

SEQ ID NO: 28 - DNA, 21 bp, Artificial Sequence
  Synthetic primer
cgtgtcgttctgggtgccgtc

SEQ ID NO: 29 - DNA, 20 bp, Artificial Sequence
  Synthetic primer
agtgctcctctgctgctcct

SEQ ID NO: 30 - DNA, 42 bp, Artificial Sequence
  Synthetic primer
ctaatacgactcactatagggcgtgtcgttctgggtgccgtc

(19) United States
(12) Patent Application Publication
(10) Pub. No.: US 2011/0287088 A1
(43) Pub. Date: Nov. 24, 2011

(54) MODULATION OF OLFML-3 MEDIATED ANGIOGENESIS

(75) Inventors: Beat A. Imhof, Geneva (CH); Marijana Miljkovic-Licina, Geneva (CH); Philippe Hammel, Geneva (CH)

(73) Assignee: RESEARCH DEVELOPMENT FOUNDATION, CARSON CITY, NV (US)

(21) Appl. No.: 13/131,491

(22) PCT Filed: Nov. 30, 2009
(86) PCT No.: PCT/US09/66054

§ 371 Date: Jul. 20, 2011

Related U.S. Application Data
(60) Provisional application No. 61/119,551, filed on Dec. 3, 2008.

(51) Int. Cl. A61K 9/127; A61K 31/7088; C07K 16/18; C07H 21/04; A61P 17/02; A61P 35/00; A61P 35/02; A61P 27/02; A61P 9/00; A61P 19/02; A61P 1/00; A61P 1/18; A61P 29/00; A61P 41/00; A61K 39/395

(52) U.S. Cl. 424/450; 424/139.1; 514/44 A; 530/387.9; 536/24.5

(57) ABSTRACT

The present invention relates to nucleic acids and antibodies against Olfml-3 and Olfml-3 protein function in angiogenesis. Angiogenesis-related conditions, such as cancer or wound healing, can be treated by the composition comprising the Olfml-3 antagonists or agonists, respectively.

The present application claims the priority benefit of U.S. provisional application No. 61/119,551, filed Dec. 3, 2008, the entire contents of which are incorporated herein by reference.

### BACKGROUND OF THE INVENTION

1. Field of the Invention

The present invention relates generally to the fields of molecular biology and oncology. More particularly, it concerns compositions comprising an inhibitory nucleic acid or an antibody for Olfml-3, a novel angiogenesis modulator, or an Olfml-3 polypeptide, and associated methods of treating angiogenesis-related conditions.

2. Description of Related Art

Angiogenesis is a multi-step cellular process of capillary sprouting and formation of neo-vasculature from preexisting blood vessels. The complex process involves disassembly of endothelial junctions, followed by endothelial cells detachment, proliferation and migration as well as subsequent re-establishment of intercellular and cell-matrix contact. As such it requires coordinated actions of a variety of vascular cell adhesion molecules and growth factors originating from endothelial cells themselves or neighboring mural cells. Indeed, angiogenesis is a tightly tuned process regulated by pro- and anti-angiogenic factors (Folkman, 1995).

Numerous studies have demonstrated that excessive angiogenesis influences significantly various disease states including tumor growth, ischemic cardiovascular pathologies or chronic inflammatory diseases (Carmeliet, 2003; Carmeliet, 2005; Gariano and Gardner, 2005).

From vascular mediated pathologies, tumor-associated angiogenesis is the most extensively studied. It was first postulated that tumors cannot grow further than a size of 2-3 mm³ in the absence of neovascularization (Folkman, 1971). Therefore, angiogenesis is a prerequisite for tumor growth and blocking this process can prevent further proliferation of tumor cells. Furthermore, prevention of angiogenesis targets normal tissue and does not escape therapy by mutagenesis as seen with tumor cells. It is thus expected that anti-angiogenic therapy be better sustained in keeping tumor growth under control than any other treatment directly addressing tumor cells. Despite the fact that vascular endothelial cell growth factor (VEGF), fibroblast growth factor (FGF) and other pro-angiogenic molecules are indispensable for vessel formations (Hanahan, 1997; Yancopoulos et al., 2000), the complete molecular and cellular mechanisms governing tumor-associated angiogenesis are poorly understood.

In addition, diseases complicated by vascular leakage and/or neovascularization in the eye are responsible for the vast majority of visual morbidity and blindness in developed countries. Retinal neovascularization occurs in ischemic retinopathies such as diabetic retinopathy and is a major cause of visual loss in working age patients (Klein et al., 1984). Choroidal neovascularization occurs as a complication of age-related macular degeneration and is a major cause of visual loss in elderly patients (Ferris et al., 1984). Improved treatments are needed to reduce the high rate of visual loss, and their development is likely to be facilitated by greater understanding of the molecular pathogenesis of ocular neovascularization.

In clinical trials, beneficial effects of anti-angiogenic drugs were so far reached with antibodies against VEGF in the context of colon and breast carcinomas. However, it was less successful with other tumors for which alternate factors may be involved. Thus, other molecules involved in angiogenesis should be identified and used alone or in combination with the growth factors. Targeting novel vascular molecules expressed and/or secreted by angiogenic endothelial cells represent an additional avenue.

On the other hand, insufficient angiogenesis is also related to a large number of diseases and conditions, such as cardiovascular diseases (e.g., coronary artery diseases) and delayed wound healing. To date, cardiovascular diseases are the leading cause of mortality in the United States, Europe, and Israel. In the United States, approximately one million deaths per year are attributed to cardiac causes, fifty percent of which are attributed to Coronary Artery Disease (CAD). The major morbidity from CAD is a result of obstructive coronary artery narrowing and the resultant myocardial ischemia CAD affects more than 13 million people, and its annual economic burden is in excess of sixty billion U.S. Dollars.

Mechanical revascularization of obstructive coronary stenoses by percutaneous techniques, including percutaneous transluminal angioplasty and stent implantation, is used to restore normal coronary artery blood flow. In addition, coronary artery occlusion bypass surgery is performed using arterial and venous conduits as grafts onto the coronary arterial tree. These treatment modalities have significant limitations in individuals with diffuse atherosclerotic disease or severe small vessel coronary artery disease, in diabetic patients, as well as in individuals who have already undergone surgical or percutaneous procedures.

For these reasons, therapeutic angiogenesis, aimed at stimulating new blood vessel growth, is highly desirable. The therapeutic concept of angiogenesis therapy is based on the premise that the existing potential for vascular growth inherent to vascular tissue can be utilized to promote the development of new blood vessels under the influence of the appropriate angiogenic molecules. Therapeutic angiogenesis defines the intervention used to treat local hypovascularity by stimulating or inducing neovascularization for the treatment of ischemic vascular disease.

### SUMMARY OF THE INVENTION

In accordance with certain aspects of the present invention, there is provided an isolated nucleic acid molecule comprising a sequence that will hybridize with an Olfml-3 mRNA sequence selected from the group consisting of SEQ ID NOs:1-7 and inhibit the expression of Olfml-3 in a cell. The nucleic acid in this regard is preferably an siRNA, a double stranded RNA, a short hairpin RNA, an antisense oligonucleotide, a ribozyme, a nucleic acid encoding thereof. Preferably, the nucleic acid is further defined as an siRNA or a nucleic acid encoding an siRNA.

C termini of Olfml-3 amino acid sequences encode highly conserved olfactomedin-like (OLFML) domain (corresponding to amino acid 137-401 in mONT3, amino acid 138-401 in rONT3, amino acid 135-401 in hONT3 and amino acid 128-388 in cONT1), which may be critical for the novel function in angiogenesis. By using methods known to an ordinary person in the art, inhibitory nucleic acid sequences such as siRNA could be designed to target Olfml-3 mRNA sequences (SEQ ID NOs: 1-7), preferably the OLFML domain-coding sequences at the C terminus, such as nucleic acid sequences encoding amino acids 342-351 (e.g., RARIQCSFDA (SEQ ID NO:18) in mONT3 and hONT3), 130-139 (e.g., DMVTDCSYT (SEQ ID NO:19) in mONT3 and DMVTDCGYT (SEQ ID NO:20) in hONT3) or 288-296 (e.g., ATRDDDRHL (SEQ ID NO:21) in mONT3 and ATREDDRHL (SEQ ID NO:22) in hONT3) of about 400 amino acids of Olfml-3 amino acid sequences (SEQ ID NOs:11-17). For example, the siRNA may comprise SEQ ID NO:8, or comprise SEQ ID NO:10, or comprise SEQ ID NOs: 8 and 10, or comprise SEQ ID NOs: 9 and 10.

Alternatively, an antibody or a fragment thereof that binds to an Olfml-3 amino acid sequence selected from SEQ ID NOs:11-17 and inhibits the activity of Olfml-3 in angiogenesis may be provided. The antibody may be selected from the group consisting of a monoclonal antibody, a polyclonal antibody, a chimeric antibody, an affinity matured antibody, a humanized antibody, and a human antibody. Preferably, the antibody is a monoclonal antibody or a humanized antibody. The antibody fragment may be a Fab, Fab′, Fab′-SH, F(ab′)₂, or scFv.

For medical or clinical application, the antibody or fragment may be attached to an agent to be targeted to an Olfml-3-expressing cell. The agent may be a cytotoxic agent, a cytokine, an anti-angiogenic agent, a chemotherapeutic agent, a diagnostic agent, an imaging agent, a radioisotope, a pro-apoptosis agent, an enzyme, a hormone, a growth factor, a peptide, a protein, an antibiotic, an antibody, a Fab fragment of an antibody, an imaging agent, an antigen, a survival factor, an anti-apoptotic agent, a hormone antagonist, a virus, a bacteriophage, a bacterium, a liposome, a microparticle, a magnetic bead, a microdevice, a cell, a nucleic acid or an expression vector.

There may also be provided a pharmaceutical composition comprising one or more nucleic acids or the antibody or fragment described above in a pharmaceutically acceptable carrier, for example, a pharmaceutical composition comprising the antibody or fragment and a pharmaceutically acceptable carrier or a pharmaceutical composition comprising one or more nucleic acids described above and a pharmaceutically acceptable carrier.

The pharmaceutical composition of the present invention may further comprise a lipid component, which is believed to likely give the nucleic acid an improved stability, efficacy and bioavailability, with perhaps even reduced toxicity. The lipid component may form a liposome, but this is not believed to be required. In certain aspects, the composition further comprises cholesterol or polyethyleneglycol (PEG).

Exemplary lipids include, but are not limited to, 1,2-dioleoyl-sn-glycero-3-phosphatidylcholine (DOPC), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), palmitoyloeoyl phosphatidylcholine (“POPC”), lysophosphatidylcholine, dilinoleoylphosphatidylcholine distearoylphophatidylethanolamine (“DSPE”), dimyristoyl phosphatidylethanolamine (“DMPE”), dipalmitoyl phosphatidylethanolamine (“DPPE”), palmitoyloeoyl phosphatidylethanolamine (“POPE”), lysophosphatidylethanolamine, phosphatidylserine, phosphatidylglycerol, dimyristoyl phosphatidylserine (“DMPS”), dipalmitoyl phosphatidylserine (“DPPS”), brain phosphatidylserine (“BPS”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), or dioleoylphosphatidylglycerol (“DOPG”).

It is contemplated that the Olfml-3 inhibitory molecules (one or more of the nucleic acids or the antibody or the fragment) or the composition of the present invention described above may be used in the treatment of any disease or disorder in which angiogenesis plays a role, which will be referred to generally as an angiogenesis-related condition. It is contemplated that the invention will find applicability in any such disorder in humans or animals. Exemplary angiogenesis-related conditions include cancer, ocular neovascularization, arterio-venous malformations, coronary restenosis, peripheral vessel restenosis, glomerulonephritis, rheumatoid arthritis, ischemic cardiovascular pathologies, chronic inflammatory diseases, etc.

In the case of cancer, exemplary angiogenic cancers include angiogenic breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia. Ocular neovascularization disorders include macular degeneration (e.g., age-related macular degeneration (AMD), corneal graft rejection, corneal neovascularization, retinopathy of prematurity (ROP) and diabetic retinopathy.

In other aspects, there may be provided a pharmaceutical composition for inducing angiogenesis in a subject, comprising an isolated Olfml-3 protein or peptide comprising at least 10 amino acids having at least 95% identity to an amino acid sequence selected from the group consisting of SEQ ID NOs:11-17; and a pharmaceutically acceptable carrier. Specifically, the isolated Olfml-3 protein or peptide comprises at least 10 amino acids having at least 95% identity to an olfactomedin-like domain selected from the group consisting of amino acids 137 to 401 of SEQ ID NO:11, amino acids 135 to 401 of SEQ ID NO:12, amino acids 138 to 401 of SEQ ID NO:13, and amino acids 128 to 388 of SEQ ID NO:14.

In certain embodiments the size of the Olfml-3 peptide having at least 95% identity to an amino acid sequence selected from the group consisting of SEQ ID NOs:11-17 may comprise, but is not limited to, about 10, about 15, about 20, about 25, about 30, about 50, about 80, about 100, about 150, about 200, about 300, about 400, and any range derivable therein. Particularly, the Olfml-3 peptide may have about 96%, 97%, 98%, 99%, 100%, or any range derivable therein identity to an amino acid sequence selected from the group consisting of SEQ ID NOs:11-17.

In a further embodiment, the composition may further comprise a lipid component, which may form a liposome. In certain aspects, the composition further comprises cholesterol or polyethyleneglycol (PEG). Exemplary lipids are described as above.

In still further embodiments, the invention is directed to a method for treating an angiogenesis-related condition comprising administering to a subject in need of angiogenesis an amount of the composition that is effective to induce angiogenesis. Preferably, the subject is a human subject. Exemplary angiogenesis-related condition in need of an angiogenesis include, but not limited to, transplantation, cardiovascular diseases, aneurisms or wound healing. In particular embodiments, the angiogenesis-related condition is wound healing.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan; however, these terms may be used interchangeably with “comprise” or “comprising” respectively.

As used herein the specification, “a” or “an” may mean one or more. As used herein in the claim(s), when used in conjunction with the word “comprising”, the words “a” or “an” may mean one or more than one. The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Differential expression of Olfml-3 mRNA in angiogenic versus resting endothelial cells. Validation of data obtained by microarray analysis using quantitative RT-PCR. Bars represent the quantity of the Olfml-3 mRNA (relative units) in total RNA isolates from angiogenic and resting cells. Values for each sample were normalized to three murine house keeping genes: β-actin, 13-tubulin and EEF1A. Relative values from individual experiments were averaged and plotted with standard deviation (SD) as error bars. The statistical analysis was performed using the Welch t-test (p=0.00918).

FIGS. 2A-C: Vascular specificity of Olfml-3 gene expression in mouse tissues. Confocal view of cryosections after double staining in situ hybridization showing co-localization of endothelial cells (blue TO-PRO nuclear stain, all panels) expressing mouse PECAM-1 (arrows) and Olfml-3 (arrows) of mouse heart (FIG. 2A, middle and right panel); LLC1 tumor (FIG. 2B, right panel) and bFGF-treated matrigel plugs (FIG. 2C, right panel). The Olfml-3 sense riboprobes, as negative controls, do not give fluorescent signal in double in situ hybridization (FIG. 2A-C, left panels).

FIG. 3: Validation of down-regulation of Olfml-3 gene expression by siRNAs. Inhibition of Olfml-3 expression in angiogenic cells by three siRNA sequences (Olfml-3 siRNA 1, 2 and 3) and their combinations (Olfml-3 siRNA 1+2, 2+3, 1+3). Transfection of Olfml-3-targeted and control (nh siRNA and GAPDH) siRNAs at the concentration of 0.5 μM was carried out using Nucleofector. At 24 hours post-transfection, expression of target and control genes were analyzed by qPCR. The values were normalized to the expression levels of mouse α-actin, β-tubulin and EEF1A. Abbreviations: nh siRNA, non homologous siRNA; GAPDH, glyceraldehyde-3-phosphate dehydrogenase; siRNAs, small interfering RNAs; qPCR, quantitative polymerase chain reaction.

FIGS. 4A-B: Delayed wound healing of endothelial cells silenced for Olfml-3 expression. FIG. 4A: Monolayer cultures of angiogenic cells from Olfml-3 silenced (Olfml-3 siRNA 3, 0.5 μM) and control (nh siRNA, 0.5 μM) angiogenic cells were wounded with a pipette tip (yellow area). Cells at the edge of the wound migrated into the wounded area. After 16 hours the cells were photographed (violet area) and the migrated distance was determined. FIG. 4B: The progress of wound closure, expressed as migrated distance (μm per 16 hours), was significantly delayed in the Olfml-3 siRNA 1+3 silenced cells (black bar) compared to control cells (dark grey bars). Values from individual experiments were averaged and the mean values were plotted with standard deviation (SD) as error bars. The statistical analysis was performed using t-test.

FIGS. 5A-E. Silencing of Olfml-3 in endothelial cells attenuates the initiation and the final steps angiogenesis in vitro. FIG. 5A: Sprout formation in vitro starts with individual endothelial cells sending out spikes (as shown by control, mock transfected cells at 24 hours, blue arrowhead). It continues with cell-cell contact formation, which leads to branching of the proliferating cells forming a polygonal network (as shown by control, mock transfected cells at 32-144 h, blue arrows). Two Olfml-3 siRNAs (Olfml-3 siRNA 1 and 3, 0.5 μM) and their combination (Olfml-3 siRNA 1+3, 0.5 μM each) were transfected into angiogenic cells, which are then cultured in 3D fibrin gels for 144 hours. During early phases of the angiogenesis assay (24-32 hours), delayed formation of spikes (arrowheads) was observed with Olfml-3-silenced cells (Olfml-3 1, 3, 1+3, 0.5 μM). During later phases of the angiogenesis assay (48-144 hours), decreased ability for branching and sprout formation was observed with silenced cells leading to a less complex network (arrows). FIG. 5B: Quantification of angiogenic cells numbers that form spikes at early phases during the angiogenesis assay. The spikes forming (dark grey bars) and not forming (light grey bars) cells during the first 24 in 3D fibrin gels were counted and plotted as percentiles. Delayed formation of spikes was observed with Olfml-3-silenced cells (Olfml-3 1, 3, 1+3, 0.5 μM), when compared with the control cells (mock and nh siRNA transfected cells). The mean and standard deviation of two experiments are shown. FIG. 5C: Quantification of angiogenic cells numbers that form spikes at early phases during the angiogenesis assay. The spikes forming (dark grey bars) and not forming (light grey bars) cells during the first 32 hours in 3D fibrin gels were counted and plotted as percentiles. Delayed formation of spikes was observed with Olfml-3-silenced cells (Olfml-3 1, 3, 1+3, 0.5 μM), when compared with the control cells (mock and nh siRNA transfected cells). The mean and standard deviation of two experiments is shown. FIG. 5D: Measurement of total surface of the vascular net representing the capillary-like network at 56 hours. Development of this network decreased when Olfml-3 gene was silenced (Olfml-3 siRNAs 1, 3, 1+3, 0.5 μM) compared to control cells (mock or nh siRNA transfected cells). FIG. 5E: Quantification of the number of apoptotic endothelial cells 6 days after seeding into 3D fibrin gels. Olfml-3 silencing did not show significant cell death (Olfml-3 siRNA 1, 3 and 1+3, 0.5 μM) when compared to control cells (mock, nh siRNA or GAPDH transfected cells, 0.5 μM).

FIG. 6: Production and purification of recombinant mouse Olfml-3-FLAG. The full length mouse Olfml-3 gene was cloned as a FLAG tagged construct into the expression vector pcDNA3.3-TOPO. Transfected MDCK cells were selected by Neomycin and the cell culture supernatant collected. The protein was then affinity purified on an anti-FLAG affinity column and eluted with FLAG peptide. Shown are Western blots (blotting) of supernatant and affinity purified protein after Immunoreactions with anti-FLAG antibodies, and SDS gel of purified protein stained by Coomassie blue. Two bands appeared probably representing different glycosylation stages.

FIG. 7: Recombinant Olfml-3 induces angiogenic sprouting of endothelial cells in fibrin gels. Olfml-3 or mock transfected MDCK cells were plated in a culture well and overlaid by a fibrin gel containing t.End.1 endothelial cells. The total length of the forming vascular skeleton was then determined using Metamorph software. Clearly, Olfml-3 secreted by MDCK cells increases vascular sprouting.

FIG. 8: Characterization of two monoclonal antibodies against mouse Olfml-3: 16F3 and 27B8. Human JAM-C-FLAG, mouse truncated JAM-C-FLAG and mouse Olfml-3-FLAG were detected by enzyme linked immunosorbent assay (ELISA) using D33 antibody recognizing human but not truncated mouse JAM-C; 16F3 or 27B8 antibodies against mouse Olfml-3 protein. Negative control (control) was irrelevant isotype-matched antibody.

FIGS. 9A-C: Treatment of mice with the anti-Olfml-3 antibodies reduces tumor growth. C57BL6/J mice were injected subcutaneously (s.c.) with Lewis lung carcinoma cells (LLC1) into the flank. Mice received intraperitoneal injections (i.p.) of either PBS, isotype-matched control antibody (ctrl mAb 64), or 16F3 and 27B8 anti-Olfml-3 antibodies every third day (200 μg, 200 μg and 50 μg, respectively). When control tumors (PBS-injected mice) reached the maximum allowed size of 0.5 cm, all tumors were excised and analyzed. FIG. 9A, macroscopic aspects of 9-days-old tumors grown in mice treated with PBS, control mAb 64 antibody, 16F3 or 27B8 anti-Olfml-3 antibody. Mice treated with 16F3 and 27B8 antibodies showed reduced tumor growth compared to controls, as evidenced by measuring tumor weight (FIG. 9B) and tumor volume (FIG. 9C). PBS (n=8), ctrl mAb 64 antibody (n=10), 16F3 (n=10) and 27B8 (n=7). Columns, mean; bars, SE; *, p<0.05. Bar, 0.5 cm.

### DESCRIPTION OF ILLUSTRATIVE EMBODIMENTS

### I. Aspects of the Present Invention

In clinical trials, beneficial effects of anti-angiogenic drugs were so far reached with antibodies against VEGF in the context of colon and breast carcinomas. However, it was less successful with other tumors for which alternate factors may be involved. Thus, other molecules involved in angiogenesis should be identified and used in combination with the growth factors. Targeting novel vascular molecules expressed and/or secreted by angiogenic endothelial cells represent an additional avenue.

The present invention is based, in part, on the finding that Olfml-3 is a novel angiogenesis modulator. For example, the inventors have found that decreased Olfml-3 expression, such as by siRNA targeting, results in reduction of migration of angiogenic cells and attenuation of initial and final steps of angiogenesis. On the other hand, Olfml-3 proteins or peptides, or associated pharmaceutical compositions and methods may be used to induce angiogenesis when needed. Aspects of the present invention can be used to prevent or treat a disease or disorder associated with Olfml-3 mediated angiogenesis. Functioning of Olfml-3 may be reduced or enhanced by any suitable drugs to stimulate or prevent angiogenesis. Such exemplary substances can be an anti-Olfml-3 antibody, soluble Olfml-3 receptors or blocking small molecules. Alternatively, the function of Olfml-3 may also be blocked by reducing its gene expression, e.g., by an siRNA approach. In certain aspects, the present invention provides compositions and methods of delivery of an inhibitory nucleic acid or antibody specific for Olfml-3 to treat angiogenesis-related disease, such as cancer. Further embodiments and advantages of the invention are described below.

### II. Olfml-3

Olfactomedin-like protein 3 (Olfml-3) is a protein that in humans is encoded by the OLFML3 gene. The inventors used the t.End.1V^{high} angiogenic and t.End.1V^{low} resting cell lines to identify novel molecules differentially expressed and associated with angiogenesis. Among the identified new angiogenesis-associated genes, which fulfill the criteria described above they identified the mouse Olfml-3 gene (olfactomedin-like 3) (synonyms: mONT3, HNOEL-iso, hOLF44).

Some olfactomedin family members are implicated in developmental processes where they play regulatory roles such as tiarin (Tsuda et al., 2002), pDP4 (Rosenbauer et al., 2004) and noelin (Moreno and Bronner-Fraser, 2005) (Barembaum et al., 2000). Gain-of-function studies have shown that Olfml-3 (mONT3) exhibits a dorsalizing effect, as shown for tiarin, when over-expressed in Xenopus embryos (Ikeya et al., 2005), suggesting its activity in Xenopus ectodermal patterning. Recently it was shown that Xenopus ONT1 is a key molucule for fine-tuning of the Chordin/bone morphogenetic protein (BMP) system, where it acts as a secreted scaffold for the B1TP-mediated degradation of chordin (Harland, 2008; Inomata et al., 2008; Sakuragi et al., 2006). This suggests that Olfml-3 may serve as scaffold for different enzymes and substrates (Tomarev and Nakaya, 2009). All these data from disease states to developmental events underline the importance of understanding the functions of olfactomedin domain-containing proteins.

Identified by phylogenetic analysis, human hOLF44 gene encodes for a secreted glycoprotein belonging to the Olfactomedin/Noelin/Tiarin family. Along with mONT2 (olfactomedin-like 1) and chick cONT1, the human Olfml-3 gene belongs to a novel, uncharacterized olfactomedin-like (ONT) subfamily of secreted molecules (Ikeya et al., 2005), including mONT3, rONT3, hONT3, cONT1, mONT2, rONT2, and hONT2. This secreted glycoprotein contains a putative signal peptide at the N-terminus, a coiled-coil domain in the middle of the sequence and an olfactomedin-like (OLF) domain at the C-terminus (Zeng et al., 2004). This molecule is involved in the formation of extracellular matrix (ECM) around olfactory neurons (Snyder et al., 1991; Yokoe and Anholt, 1993) and has regulatory role in vertebrate neural development (Barembaum et al., 2000; Tsuda et al., 2002).

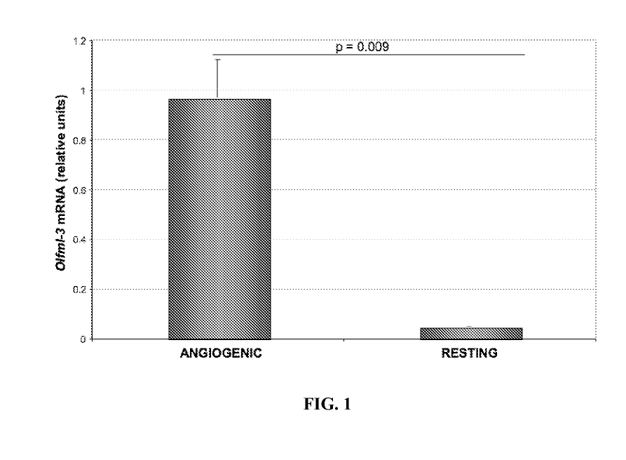

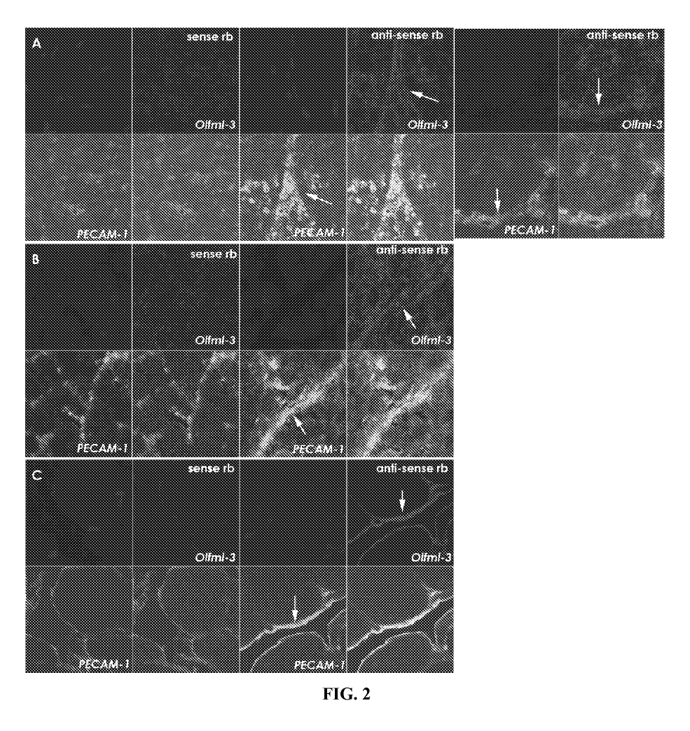

The olfactomedin-like (ONT) subfamily is distinct from the olfactomedin (OLF) subfamily consisting of well-characterized members such as olfactomedin. The phylogenetic analysis (FIG. 1 of Ikeya et al., 2005) revealed the olfactomedin-like domains are highly conserved among this subfamily of olfactomedin-like proteins with more than 90% homology in the mouse, rat and human counterparts of ONT3 (Olfml-3) and at lesser extent (64%) in the chicken cONT1 (Olfml-3). However, the homology of the olfactomedin-like domains to the olfactomedin domains of noelin, tiarin or other olfactomedin family members is as low as about 30% (see FIG. 1B, Ikeya et al., 2005).

The highest level of human Olfml-3 (hOLF44) mRNA expression was found in placenta, but also in liver and heart, though at lower expression levels (Zeng et al., 2004). Endogenous hOLF44 was found in the extracellular space surrounding syncytiotrophoblastic cells on the fetal side of human term placenta, demonstrating that the molecule was secreted (Zeng et al., 2004). Tagged recombinant hOLF44 protein enriched in perinuclear regions of COS-7 cells, most likely in the endoplasmic reticulum providing evidence that it may take the classical secretory pathway (Zeng et al., 2004). These findings suggest a role for human Olfml-3 as a component associated to extracellular matrix (ECM) possibly implicated in matrix-related placental and embryonic development or similar processes (Zeng et al., 2004).

The rat orthologue of the human Olfml-3 (the rat HNOEL-iso) gene was found to be expressed in iris, sclera, the trabecular meshwork of the retina and the optic nerve (Ahmed et al., 2004). Expression of the mouse counterpart of the human Olfml-3 (mONT3) gene was detected very early during embryogenesis: firstly, in the proximal regions of the alantois, subsequently in the presumptive lateral mesoderm plate and than in the CNS and heart on embryonic day E 8.5 (Ikeya et al., 2005). The mONT-3 knock-out mice (male and female) were found to be viable, normal and fertile, suggesting that mONT3 is dispensable for normal embryogenesis and compensated by other family members (Ikeya et al., 2005). Moreover, gain-of-function studies showed mONT3 exhibits a dorsalizing effect, when over-expressed in Xenopus embryos (Ikeya et al., 2005) suggesting a role in embryonic patterning. However, putative involvement of the Olfml-3 gene in angiogenesis has never been demonstrated until the present invention.

### III. Inhibition of Olfml-3 Expression

As mentioned, the present invention contemplates in certain aspects the use of one or more inhibitory nucleic acid for inhibiting or reducing the angiogenic action of Olfml-3. Using bioinformatics work and experimental approaches the inventors have analyzed the expression of glycoprotein Olfml-3 in mouse angiogenic cells. Olfml-3 is a soluble molecule of 406 amino-acids with an orphan receptor. The Olfml-3 gene is highly expressed by angiogenic but not resting endothelial cells and its expression is driven by angiogenic growth factors. The inventors analyzed the expression pattern of the Olfml-3 gene in several mouse tissues. Vascular specificity of its expression was found in quiescent blood vessels of highly vascularized organs. More importantly, increased vascular expression of the Olfml-3 gene in vivo was detected inhighly proliferative, angiogenic tumor tissue and in new blood vessels induced by the angiogenic factor bFGF in matrigel plugs. Vascular specificity of the Olfml-3 gene and its high level of expression in new blood vessels suggests that mOlfml-3 expression is associated with vascular patterning in normal and pathological angiogenesis.

A. Inhibitory Nucleic Acids

Examples of an inhibitory nucleic acid include but are not limited to siRNA (small interfering RNA), short hairpin RNA (shRNA), double-stranded RNA, an antisense oligonucleotide, a ribozyme and a nucleic acid encoding thereof. An inhibitory nucleic acid may inhibit the transcription of a gene or prevent the translation of a gene transcript in a cell. An inhibitory nucleic acid may be from 16 to 1000 nucleotides long, and in certain embodiments from 18 to 100 nucleotides long. In certain embodiments, the inhibitory nucleic acid is an isolated nucleic acid that binds or hybridizes to a Olfml-3 mRNA sequence selected from the group consisting of SEQ ID NOs:1-7 and inhibits the expression of a gene that encodes Olfml-3

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory nucleic acids are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

B. Preparation of siRNA

Since the discovery of RNAi by Fire and colleagues in 1998, the biochemical mechanisms have been rapidly characterized. Long double stranded RNA (dsRNA) is cleaved by Dicer, which is an RNAase III family ribonuclease. This process yields siRNAs of ˜21 nucleotides in length. These siRNAs are incorporated into a multiprotein RNA-induced silencing complex (RISC) that is guided to target mRNA. RISC cleaves the target mRNA in the middle of the complementary region.

In mammalian cells, the related microRNAs (miRNAs) are found that are short RNA fragments (˜22 nucleotides). mRNAs are generated after Dicer-mediated cleavage of longer (˜70 nucleotide) precursors with imperfect hairpin RNA structures. The miRNA is incorporated into an miRNA-protein complex (miRNP), which leads to translational repression of target mRNA.

In designing RNAi there are several factors that need to be considered such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Particularly the siRNA exhibits greater than 80, 85, 90, 95, 98% or even 100% identity between the sequence of the siRNA and a portion of Olfml-3 mRNA sequence. Sequences less than about 80% identical to the target gene are substantially less effective. Thus, the greater identity between the siRNA and the Olfml-3 gene to be inhibited, the less likely expression of unrelated genes will be affected.

In addition, the size of the siRNA is an important consideration. In some embodiments, the present invention relates to siRNA molecules that include at least about 19-25 nucleotides, and are able to modulate Olfml-3 gene expression. In the context of the present invention, the siRNA is particularly less than 500, 200, 100, 50 or 25 nucleotides in length. More particularly, the siRNA is from about 19 nucleotides to about 25 nucleotides in length.

To improve the effectiveness of siRNA-mediated gene silencing, guidelines for selection of target sites on mRNA have been developed for optimal design of siRNA (Soutschek et al., 2004; Wadhwa et al., 2004). These strategies may allow for rational approaches for selecting siRNA sequences to achieve maximal gene knockdown. To facilitate the entry of siRNA into cells and tissues, a variety of vectors including plasmids and viral vectors such as adenovirus, lentivirus, and retrovirus have been used (Wadhwa et al., 2004).

Within an inhibitory nucleic acid, the components of a nucleic acid need not be of the same type or homogenous throughout (e.g., an inhibitory nucleic acid may comprise a nucleotide and a nucleic acid or nucleotide analog). Typically, an inhibitory nucleic acid form a double-stranded structure; the double-stranded structure may result from two separate nucleic acids that are partially or completely complementary. In certain embodiments of the present invention, the inhibitory nucleic acid may comprise only a single nucleic acid (polynucleotide) or nucleic acid analog and form a double-stranded structure by complementing with itself (e.g., forming a hairpin loop). The double-stranded structure of the inhibitory nucleic acid may comprise 16-500 or more contiguous nucleobases, including all ranges therebetween. The inhibitory nucleic acid may comprise 17 to 35 contiguous nucleobases, more particularly 18 to 30 contiguous nucleobases, more particularly 19 to 25 nucleobases, more particularly 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

siRNA can be obtained from commercial sources, natural sources, or can be synthesized using any of a number of techniques well-known to those of ordinary skill in the art. For example, commercial sources of predesigned siRNA include Invitrogen's Stealth™ Select technology (Carlsbad, Calif.), Ambion® (Austin, Tex.), and Qiagen® (Valencia, Calif.). An inhibitory nucleic acid that can be applied in the compositions and methods of the present invention may be any nucleic acid sequence that has been found by any source to be a validated downregulator of a Olfml-3.

In one aspect, the invention generally features an isolated siRNA molecule of at least 19 nucleotides, having at least one strand that is substantially complementary to at least ten but no more than thirty consecutive nucleotides of a nucleic acid that encodes Olfml-3, and that reduces the expression of Olfml-3. In a particular embodiment of the present invention, the siRNA molecule has at least one strand that is substantially complementary to at least ten but no more than thirty consecutive nucleotides of the mRNA that encodes Olfml-3.

In another particular embodiment, the siRNA molecule is at least 75, 80, 85, or 90% homologous, particularly at least 95%, 99%, or 100% similar or identical, or any percentages in between the foregoing (e.g., the invention contemplates 75% and greater, 80% and greater, 85% and greater, and so on, and said ranges are intended to include all whole numbers in between), to at least 10 contiguous nucleotides of any of the nucleic acid sequences encoding a full-length Olfml-3 protein. Generally speaking, it is preferred that the sequence must only be sufficiently similar to permit the siRNA molecule to bind to the Olfml-3 mRNA target intracellularly, form an RISC complex, and thereby effect downregulation of expression.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 20040019001 and U.S. Pat. No. 6,673,611 (each of which is incorporated by reference in its entirety). Collectively, all such altered nucleic acids or RNAs described above are referred to as modified siRNAs.

Particularly, RNAi is capable of decreasing the expression of Olfml-3 by at least 10%, 20%, 30%, or 40%, more particularly by at least 50%, 60%, or 70%, and most particularly by at least 75%, 80%, 90%, 95% or more or any ranges in between the foregoing.

Introduction of siRNA into cells can be achieved by methods known in the art, including for example, microinjection, electroporation, or transfection of a vector comprising a nucleic acid from which the siRNA can be transcribed. Alternatively, a siRNA can be directly introduced into a cell in a form that is capable of binding to target Olfml-3 mRNA transcripts. To increase durability and membrane-permeability the siRNA may be combined or modified with liposomes, poly-L-lysine, lipids, cholesterol, lipofectine or derivatives thereof. In certain aspects cholesterol-conjugated siRNA can be used (see, Song et al., 2003).

C. Hybridization

As used herein, “hybridization”, “hybridize(s)” or “capable of hybridizing” is understood to mean the forming of a double or triple stranded molecule or a molecule with partial double or triple stranded nature. The term “anneal” or “bind” as used herein is synonymous with “hybridize.” Preferably, hybridization encompasses intracellular conditions, i.e., inhibitory nucleic acids hybridize with Olfml-3 mRNA sequences in a cell or under intracellular conditions, preferably, an angiogenic cell, and more preferably, an angiogenic cell in a subject in need of angiogenesis treatment. Intracellular conditions refer to conditions such as temperature, pH and salt concentrations typically found inside a cell, e.g., a mammalian cell, which are well know to those of ordinary skill in the art, for example,

The term “hybridization”, “hybridize(s)” or “capable of hybridizing” also encompasses the terms “stringent condition(s)” or “high stringency” and the terms “low stringency” or “low stringency condition(s).” A polynucleotide which hybridizes under an intracellular condition in the invention may for example be a polynucleotide which hybridizes under a stringent condition described below.

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are particularly for applications requiring high selectivity. Non-limiting applications include isolating a nucleic acid, such as a gene or a nucleic acid segment thereof, or detecting at least one specific mRNA transcript or a nucleic acid segment thereof, and the like.

The stringent condition may for example be a condition involving 2×SSC, 1×Denhart's solution at about 60° C. Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is particular to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

### IV. Therapeutic Antibodies

In certain embodiments, an antibody or a fragment thereof that binds to at least a portion of Olfml-3 protein and inhibits Olfml-3 activity in angiogenesis and its associated use in treatment of diseases are contemplated.

The antibody may be selected from the group consisting of a chimeric antibody, an affinity matured antibody, a polyclonal antibody, a monoclonal antibody or a humanized antibody, and a human antibody. Preferably, the anti-Olfml-3 antibody is a monoclonal antibody or a humanized antibody.

In one embodiment, the antibody is a chimeric antibody, for example, an antibody comprising antigen binding sequences from a non-human donor grafted to a heterologous non-human, human or humanized sequence (e.g., framework and/or constant domain sequences). In one embodiment, the non-human donor is a mouse. In one embodiment, an antigen binding sequence is synthetic, e.g., obtained by mutagenesis (e.g., phage display screening, etc.). In one embodiment, a chimeric antibody of the invention has murine V regions and human C region. In one embodiment, the murine light chain V region is fused to a human kappa light chain. In one embodiment, the murine heavy chain V region is fused to a human IgG1 C region.

Examples of antibody fragments suitable for the present invention include, without limitation: (i) the Fab fragment, consisting of VL, VH, CL and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker which allows the two domains to associate to form a binding domain; (viii) bi-specific single chain Fv dimers (see U.S. Pat. No. 5,091,513) and (ix) diabodies, multivalent or multispecific fragments constructed by gene fusion (US Patent App. Pub. 20050214860). Fv, scFv or diabody molecules may be stabilized by the incorporation of disulphide bridges linking the VH and VL domains. Minibodies comprising a scFv joined to a CH3 domain may also be made (Hu et al, 1996).

Olfml-3 mRNA sequences (SEQ ID NOs: 1-7) may be used to produce recombinant proteins and peptides as well known to people skilled in the art or as described in detail in the next section. For example, such mRNA sequences could be engineered into a suitable expression system, e.g., yeast, insect cells or mammalian cells, for production of an Olfml-3 protein or peptide comprising at least 10 amino acids having at least 95% identity to an amino acid sequence selected from the group consisting of SEQ ID NOs:11-17.

Animals may be inoculated with an antigen, such as a Olfml-3 protein or peptide, in order to produce antibodies specific for an Olfml-3 protein or peptides having a sequence selected from the group consisting of SEQ ID NOs:11-17. Frequently an antigen is bound or conjugated to another molecule to enhance the immune response. As used herein, a conjugate is any peptide, polypeptide, protein or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because all antibody producing cells are derived from a single B-lymphocyte cell line. Hybridoma technology involves the fusion of a single B lymphocyte from a mouse previously immunized with a Olfml-3 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced. However, in therapeutic applications a goal of hybridoma technology is to reduce the immune reaction in humans that may result from administration of monoclonal antibodies generated by the non-human (e.g. mouse) hybridoma cell line.

Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDR is derived from mouse monoclonal antibodies, and the framework regions are derived from human amino acid sequences. It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

It is possible to create engineered antibodies, using monoclonal and other antibodies and recombinant DNA technology to produce other antibodies or chimeric molecules which retain the antigen or epitope specificity of the original antibody, i.e., the molecule has a binding domain. Such techniques may involve introducing DNA encoding the immunoglobulin variable region or the CDRs of an antibody to the genetic material for the framework regions, constant regions, or constant regions plus framework regions, of a different antibody. See, for instance, U.S. Pat. Nos. 5,091,513, and 6,881,557, which are incorporated herein by this reference.

By known means as described herein, polyclonal or monoclonal antibodies, antibody fragments and binding domains and CDRs (including engineered forms of any of the foregoing), may be created that are specific to Olfml-3 protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by this reference. These techniques are further described in: Marks (1992); Stemmer (1994); Gram et al. (1992); Barbas et al. (1994); and Schier et al. (1996).

Methods for producing polyclonal antibodies in various animal species, as well as for producing monoclonal antibodies of various types, including humanized, chimeric, and fully human, are well known in the art and highly predictable. Methods for producing these antibodies are also well known and predictable. For example, the following U.S. patents and patent applications provide enabling descriptions of such methods and are herein incorporated by reference: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

It is fully expected that antibodies to Olfml-3 will have the ability to neutralize or counteract the effects of the Olfml-3 regardless of the animal species, monoclonal cell line or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response and, thus, antibodies without Fc may be preferential for prophylactic or therapeutic treatments. As described above, antibodies may also be constructed so as to be chimeric, partially or fully human, so as to reduce or eliminate the adverse immunological consequences resulting from administering to an animal an antibody that has been produced in, or has sequences from, other species.

### V. Olfml-3 Protein or Peptides

In certain aspects, the invention is directed to a pharmaceutical composition for inducing or promoting angiogenesis comprising an Olfml-3 full-length protein, or a peptide or polypeptide derived there from. SEQ ID NO:11 shows the translated product of SEQ ID NO:1 (cDNA of mouse Olfml-3). It is contemplated that the compositions and methods disclosed herein may be utilized to express all or part of sequences selected from the group consisting of SEQ ID NOs:11-17 and derivatives thereof, particularly the human Olfml-3 protein as depicted in SEQ ID NO:11. Determination of which protein or DNA molecules induce angiogenesis may be achieved using functional assays, such as measuring wound healing, which are familiar to those of skill in the art. The structure of the various polypeptides or peptides can be modeled or resolved by computer modeling, NMR, or x-ray crystallography. Such structures may be used to engineer derivatives of the various Olfml-3 protein.

A. Variants of Olfml-3 Polypeptides

Embodiments of the invention include various Olfml-3 polypeptides, peptides, and derivatives thereof. The term “biologically functional equivalent” is well understood in the art and is further defined in detail herein. Accordingly, Olfml-3 polypeptides or peptides include sequences that have between about 70% and about 80%; or more preferably, between about 81% and about 90%; or even more preferably, between about 91% and about 99%; or even more preferably, between about 95% and about 99%; of amino acids that are identical or functionally equivalent to the amino acids of Olfml-3 polypeptides selected from the group consisting of SEQ ID NOs:11-17, provided the biological activity of the protein or peptide is maintained.

The term “functionally equivalent codon” is used herein to refer to codons that encode the same amino acid, such as the six codons for arginine or serine, and also refers to codons that encode biologically equivalent amino acids as well known to people in the art.

Certain embodiments of the invention include various peptides or polypeptides of the Olfml-3 protein. For example, all or part of a Olfml-3 protein as set forth in SEQ ID NOs:11-17 may be used in various embodiments of the invention. In certain embodiments, a fragment of the Olfml-3 protein or a Olfml-3 peptide may comprise, but is not limited to at least 10, 12, 15, 20, 25, 100 amino acids and any range derivable therein.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological activity (e.g., pro-angiogenesis activity) where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of an Olfml-3 polypeptide or peptide to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA or RNA coding sequence, and nevertheless produce a protein with like properties. It is thus contemplated by the inventors that various changes may be made in the DNA or RNA sequences of genes or coding regions without appreciable loss of their biological utility or activity, as discussed herein.

In certain embodiments, an Olfml-3 polypeptide may be a fusion protein. Fusion proteins may alter the characteristics of a given polypeptide, such cellular uptake and/or permeability, antigenicity or purification characteristics. A fusion protein is a specialized type of insertional variant. This molecule generally has all or a substantial portion of the native molecule or peptide, linked at the N- or C-terminus, to all or a portion of a second polypeptide. For example, fusions typically employ leader or targeting sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals, or transmembrane regions.

B. Peptides

In this application, the products of the present invention are referred to by various terms, including “analogs,” “mimetics,” “peptidomimetics,” and “derivatives.” These terms are used interchangeably and denote equivalent compounds. Mimetics of the present invention comprise a structure which comprises a sequence or mimics the structure of a sequence set forth as SEQ ID NOs:11-17, and thus may comprise additional elements such as R-group substituents and a linker selected from the possibilities set forth in the instant invention.

As defined by the present invention, biological activity refers to the biological activity of Olfml-3 and its segments, for example, a novel activity in angiogenesis discovered by the inventors.

Mimetics of the invention may include peptide derivatives or peptide analogs and their derivatives, such as C-terminal hydroxymethyl derivatives, O-modified derivatives, N-terminally modified derivatives including substituted amides such as alkylamides and hydrazides and compounds in which a C-terminal residue is replaced with a phenethylamide analogue, glycosylated peptide derivatives, polyethylene glycol modified derivatives, or biotinylated derivatives. Peptide analogs of the invention include pharmaceutically acceptable salts of an analog.

In one aspect of the invention, the peptide analogs of the invention may be coupled directly or indirectly to at least one modifying group. In some aspects of the invention, the term “modifying group” is intended to include structures that are directly attached to the peptidic structure (e.g., by covalent bonding or covalent coupling), as well as those that are indirectly attached to the peptidic structure (e.g., by a stable non-covalent bond association or by covalent coupling through a linker to additional amino acid residues). In other aspects of the invention the term “modifying group” may also refer to mimetics, analogues or derivatives thereof. Alternatively, the modifying group can be coupled to a side chain of at least one amino acid residue of a Olfml-3 peptide, or a peptidic or a peptidomimetic. In other aspects, modifying groups covalently coupled to the peptidic structure can be attached by means and using methods well known in the art for linking chemical structures.

In one embodiment of the invention, peptides and peptide analogs are designed by replacing all or part of a structural domain with a linker or a compound that mimic such structure. In a different embodiment, all or a portion of the amino-terminal domain and all or a portion of the carboxy-terminal domain of a peptide or peptide analog are connected with a linker. In another embodiment, the peptide and peptide analogs are designed so that there are cyclized by covalent modification between residues of the peptide. A peptide analog compound of the invention may be further modified to alter the specific properties of the compound while retaining the desired functionality of the compound. For example, in one embodiment, the compound may be modified to alter a pharmacokinetic property of the compound, such as in vivo stability, solubility, bioavailability or half-life. The compound may be modified to label the compound with a detectable substance. The compound may be modified to couple the compound to an additional therapeutic moiety. To further chemically modify the compound, such as to alter its pharmacokinetic properties, reactive groups can be derivatized.

In an alternative chemical modification, a peptide analog compound of the invention may be prepared in a “prodrug” form, wherein the compound itself does not act as a peptide analog agonist, but rather is capable of being transformed, upon metabolism in vivo, into a peptide analog agonist or antagonist compound.

Mimetics of the invention may be prepared by standard techniques known in the art. A peptide or polypeptide component of an analog may comprise, at least in part, a peptide synthesized using standard techniques. Automated peptide synthesizers are commercially available. Peptides and polypeptides may be assayed for activity in accordance with methods exemplified herein. Peptides and polypeptides may be purified by HPLC and analyzed by mass spectrometry.

The analogs of the invention include peptide or polypeptide sequences wherein one or more of the amino acids have been replaced by a conservative amino acid substitution. The term “conservative amino acid substitution” refers to a peptide chain in which one of the amino acid residues is replaced with an amino acid residue having a side chain with similar properties. Families of amino acid residues having side chains with similar properties are well known in the art. These families include amino acids with acidic side chains (e.g., aspartic acid, glutamic acid), basic side chains (e.g., lysine, arginine, histidine), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

C. In Vitro Production of Olfml-3 Polypeptides or Peptides

Various types of expression vectors are known in the art that can be used for the production of protein or peptide products. For example, following transfection with a expression vector comprising a coding sequence selected from the group consisting of SEQ ID NOs:1-7 to a cell in culture, e.g., a primary mammalian cell, a recombinant Olfml-3 protein product may be prepared in various ways. A host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed. In order for the cells to be kept viable while in vitro and in contact with the expression construct, it is necessary to ensure that the cells maintain contact with the correct ratio of oxygen and carbon dioxide and nutrients but are protected from microbial contamination.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large-scale production of cells and cell products. However, suspension cultured cells have limitations, such as tumorigenic potential and lower protein production than adherent cells.

In further aspects of the invention, other protein production methods known in the art may be used, including but not limited to prokaryotic, yeast, and other eukaryotic hosts such as insect cells and the like.

Because of their relatively small size, the Olfml-3 peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences can be readily synthesized and then screened in screening assays designed to identify biologically functional equivalent peptides.

D. Protein Purification

It may be desirable to purify or isolate Olfml-3 polypeptides and peptides, or variants and derivatives thereof. Protein purification techniques are well known to those of skill in the art. These techniques involve, at one level, the crude fractionation of the cellular milieu to polypeptide and non-polypeptide fractions. Having separated the Olfml-3 polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, hydrophobic interaction chromatography, exclusion chromatography; polyacrylamide gel electrophoresis; isoelectric focusing. A particularly efficient method of purifying peptides is fast protein liquid chromatography (FPLC).

Certain aspects of the present invention concern the purification, and in particular embodiments, the substantial purification, of an encoded protein or peptide. The term “isolated or purified protein or peptide” as used herein, is intended to refer to a composition, isolatable from other components, wherein the protein or peptide is purified to any degree relative to its naturally obtainable state. A isolated or purified protein or peptide therefore also refers to a protein or peptide, free from the environment in which it may naturally occur.

Generally, “isolated or purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme.

### VI. Lipid Preparations

In certain aspects, the present invention provides methods and compositions for associating an inhibitory nucleic acid that inhibits the expression of Olfml-3, such as an siRNA, or an inhibitory antibody or a fragment thereof, or an Olfml-3 protein or peptide, with a lipid and/or liposome. The inhibitory nucleic acid may be encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The liposome or liposome/siRNA associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. An example is the lipid dioleoylphosphatidylcholine (DOPC).

“Liposome” is a generic term encompassing a variety of unilamellar, multilamellar, and multivesicular lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, certain aspects of the present invention also encompasses compositions that have different structures in solution than the normal vesicular structure. For example, the lipids may assume a micellar structure or merely exist as non-uniform aggregates of lipid molecules. Also contemplated are lipofectamine-nucleic acid complexes.

Liposome-mediated polynucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the lipid may be associated with a hemagglutinating virus (HVJ). This has been shown to facilitate fusion with the cell membrane and promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. In that such expression vectors have been successfully employed in transfer of a polynucleotide in vitro and in vivo, then they are applicable for the present invention.

Exemplary lipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, dilinoleoylphosphatidylcholine, phosphatidylcholines, phosphatidylglycerols, phosphatidylethanolamines, cholesterol.

Liposomes and lipid compositions of the present invention can be made by different methods. For example, a nucleotide (e.g., siRNA) may be encapsulated in a neutral liposome using a method involving ethanol and calcium (Bailey and Sullivan, 2000). The size of the liposomes varies depending on the method of synthesis. A liposome suspended in an aqueous solution is generally in the shape of a spherical vesicle, and may have one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Lipids suitable for use according to the present invention can be obtained from commercial sources. For example, dimyristyl phosphatidylcholine (“DMPC”) can be obtained from Sigma Chemical Co., dicetyl phosphate (“DCP”) can be obtained from K & K Laboratories (Plainview, N.Y.); cholesterol (“Chol”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform may be used as the only solvent since it is more readily evaporated than methanol.

Liposomes within the scope of the present invention can be prepared in accordance with known laboratory techniques. In certain embodiments, liposomes are prepared by mixing liposomal lipids, in a solvent in a container (e.g., a glass, pear-shaped flask). The container will typically have a volume ten-times greater than the volume of the expected suspension of liposomes. Using a rotary evaporator, the solvent may be removed at approximately 40° C. under negative pressure. The solvent may be removed within about 5 minutes to 2 hours, depending on the desired volume of the liposomes. The composition can be dried further in a desiccator under vacuum. Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

Liposomes can also be prepared in accordance with other known laboratory procedures: the method of Bangham et al. (1965), the contents of which are incorporated herein by reference; the method of Gregoriadis (1979), the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

Dried lipids or lyophilized liposomes may be dehydrated and reconstituted in a solution of inhibitory peptide and diluted to an appropriate concentration with a suitable solvent (e.g., DPBS). The mixture may then be vigorously shaken in a vortex mixer. Unencapsulated nucleic acid may be removed by centrifugation at 29,000 g and the liposomal pellets washed. The washed liposomes may be resuspended at an appropriate total phospholipid concentration (e.g., about 50-200 mM). The amount of nucleic acid encapsulated can be determined in accordance with standard methods. After determination of the amount of nucleic acid encapsulated in the liposome preparation, the liposomes may be diluted to appropriate concentrations and stored at 4° C. until use.

### VII. Treatment of Diseases

Certain aspects of the present invention can be used to prevent or treat a disease or disorder associated with Olfml-3 mediated angiogenesis. Functioning of Olfml-3 may be reduced or enhanced by any suitable drugs to stimulate or prevent angiogenesis. Such exemplary substances can be an anti-Olfml-3 antibody, soluble Olfml-3 receptors or blocking small molecules. Alternatively, the function of Olfml-3 may also be blocked by reducing its gene expression e.g. by an siRNA approach.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of a nucleic acid that inhibits the expression of a gene that encodes a Olfml-3 and a lipid for the purposes of minimizing the growth or invasion of a tumor, such as a colorectal cancer.

A “subject” refers to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

Certain aspects of the present invention may be used to treat any condition or disease associated with increased or decreased expression of a Olfml-3. For example, the disease may be an angiogenesis-related condition or disease. Angiogenesis-related condition or disease is a consequence of an imbalanced angiogenic process resulting in an excessive amount of new blood vessels or insufficient number of blood vessels.

In certain embodiments, the present methods can be used to inhibit angiogenesis which is non-pathogenic; i.e., angiogenesis which results from normal processes in the subject. Examples of non-pathogenic angiogenesis include endometrial neovascularization, and processes involved in the production of fatty tissues or cholesterol. Thus, the invention provides a method for inhibiting non-pathogenic angiogenesis, e.g., for controlling weight or promoting fat loss, for reducing cholesterol levels, or as an abortifacient.

The present methods can also inhibit angiogenesis which is associated with an angiogenic disease; i.e., a disease in which pathogenicity is associated with inappropriate or uncontrolled angiogenesis. For example, most cancerous solid tumors generate an adequate blood supply for themselves by inducing angiogenesis in and around the tumor site. This tumor-induced angiogenesis is often required for tumor growth, and also allows metastatic cells to enter the bloodstream.

Other angiogenic diseases include diabetic retinopathy, age-related macular degeneration (ARMD), psoriasis, rheumatoid arthritis and other inflammatory diseases. These diseases are characterized by the destruction of normal tissue by newly formed blood vessels in the area of neovascularization. For example, in ARMD, the choroid is invaded and destroyed by capillaries. The angiogenesis-driven destruction of the choroid in ARMD eventually leads to partial or full blindness. The angiogenesis-related conditions also include ocular neovascularization, arterio-venous malformations, coronary restenosis, peripheral vessel restenosis, glomerulonephritis, rheumatoid arthritis, ischemic cardiovascular pathologies, or chronic inflammatory diseases.

Exemplary eye angiogenic diseases to be treated or prevented also include choroidal neovascularization (CNV) due to any cause including but not limited to age-related macular degeneration, ocular histoplasmosis, pathologic myopia, and angioid streaks. It also applies to retinal neovascularization of any cause including but not limited to proliferative diabetic retinopathy, retinal vein occlusions, and retinopathy of prematurity. It also applies to iris neovascularization and corneal neovascularization of any causes.

The neovascularization may also be neovascularization associated with an ocular wound. For example, the wound may the result of a traumatic injury to the globe, such as a corneal laceration. Alternatively, the wound may be the result of ophthalmic surgery. In some embodiments, the methods of the present invention may be applied to prevent or reduce the risk of proliferative vitreoretinopathy following vitreoretinal surgery, prevent corneal haze following corneal surgery (such as corneal transplantation and laser surgery), prevent closure of a trabeculectomy, prevent or substantially slow the recurrence of pterygii, and so forth.

The neovascularization may be located either on or within the eye of the subject. For example, the neovascularization may be corneal neovascularization (either located on the corneal epithelium or on the endothelial surface of the cornea), iris neovascularization, neovascularization within the vitreous cavity, retinal neovasculization, or choroidal neovascularization. The neovascularization may also be neovascularization associated with conjunctival disease.

Particulary, a siRNA that binds to a nucleic acid that encodes a Olfml-3 may be administered to treat a cancer. The cancer may be a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Nonetheless, it is also recognized that certain aspects of the present invention may also be used to treat a non-cancerous disease (e.g., a fungal infection, a bacterial infection, a viral infection, and/or a neurodegenerative disease).

In certain embodiments, Olfml-3 protein or peptide is contemplated to treat angiogenesis-related conditions in a subject in need of angiogenesis. Insufficient angiogenesis is related to a large number of diseases and conditions, such as cardiovascular diseases, transplantation, aneurisms and delayed wound healing. Therapeutic angiogenesis is aimed at stimulating new blood vessel growth. The concept of such a therapy is based on the premise that the inherent potential of vascularization in a vascular tissue can be utilized to promote the development of new blood vessels under the influence of the appropriate angiogenic molecules.

### VIII. Pharmaceutical Preparations

Where clinical application of a composition containing an inhibitory nucleic acid is undertaken, it will generally be beneficial to prepare a pharmaceutical composition appropriate for the intended application. This will typically entail preparing a pharmaceutical composition that is essentially free of pyrogens, as well as any other impurities that could be harmful to humans or animals. One may also employ appropriate buffers to render the complex stable and allow for uptake by target cells.

The phrases “pharmaceutical or pharmacologically acceptable” refers to molecular entities and compositions that do not produce an adverse, allergic or other untoward reaction when administered to an animal, such as a human, as appropriate. The preparation of a pharmaceutical composition comprising a inhibitory nucleic acid or additional active ingredient will be known to those of skill in the art in light of the present disclosure, as exemplified by Remington (2005), incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is particularly formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 microgram/kg/body weight to about 500 milligram/kg/body weight, etc., can be administered.

A gene expression inhibitor may be administered in a dose of 1-100 (this such range includes intervening doses) or more μg or any number in between the foregoing of nucleic acid per dose. Each dose may be in a volume of 1, 10, 50, 100, 200, 500, 1000 or more μl or ml or any number in between the foregoing.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

In particular embodiments, the compositions of the present invention are suitable for application to mammalian eyes. For example, the formulation may be a solution, a suspension, or a gel. In some embodiments, the composition is administered via a biodegradable implant, such as an intravitreal implant or an ocular insert, such as an ocular insert designed for placement against a conjunctival surface. In some embodiments, the therapeutic agent coats a medical device or implantable device.

In preferred aspects the formulation of the invention will be applied to the eye in aqueous solution in the form of drops. These drops may be delivered from a single dose ampoule which may preferably be sterile and thus rendering bacteriostatic components of the formulation unnecessary. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or similar vehicle which forms dissolvable inserts that are placed beneath the eyelids.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, or respiratory tract, aerosol delivery can be used. Volume of the aerosol is between about 0.01 ml and 0.5 ml.

An effective amount of the therapeutic composition is determined based on the intended goal. For example, one skilled in the art can readily determine an effective amount of the siRNA of the invention to be administered to a given subject, by taking into account factors such as the size and weight of the subject; the extent of the neovascularization or disease penetration; the age, health and sex of the subject; the route of administration; and whether the administration is regional or sysemic. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

Precise amounts of the therapeutic composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### IX. Combination Treatments

In certain embodiments, the compositions and methods of the present invention involve an inhibitor of expression of Olfml-3, or construct capable of expressing an inhibitor of Olfml-3 expression, or an antibody or an antibody fragment against Olfml-3 to inhibit its activity in angiogenesis, in combination with a second or additional therapy. Such therapy can be applied in the treatment of any disease that is associated with increased expression or activity of a Olfml-3. For example, the disease may be an angiogenesis-related disease.

The methods and compositions including combination therapies enhance the therapeutic or protective effect, and/or increase the therapeutic effect of another anti-angiogenesis, anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an inhibitor of gene expression and a second therapy. A tissue, tumor, or cell can be contacted with one or more compositions or pharmacological formulation(s) including one or more of the agents (i.e., inhibitor of gene expression or an anti-cancer agent), or by contacting the tissue, tumor, and/or cell with two or more distinct compositions or formulations, wherein one composition provides 1) an inhibitor of gene expression; 2) an anti-cancer agent, or 3) both an inhibitor of gene expression and an anti-cancer agent. Also, it is contemplated that such a combination therapy can be used in conjunction with a chemotherapy, radiotherapy, surgical therapy, or immunotherapy.

An inhibitor of gene expression and/or activity may be administered before, during, after or in various combinations relative to an anti-cancer treatment. The administrations may be in intervals ranging from concurrently to minutes to days to weeks. In embodiments where the inhibitor of gene expression is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the inhibitor of gene expression therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6 or 7) to several weeks (1, 2, 3, 4, 5, 6, 7 or 8) lapse between respective administrations.

In certain embodiments, a course of treatment will last 1-90 days, or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc.

Various combinations may be employed. For the example below an inhibitor of gene expression therapy is “A” and an anti-cancer therapy is “B”:

A/B/AB/A/BB/B/AA/A/BA/B/BB/A/AA/B/B/BB/A/B/B

B/B/B/A B/B/A/B A/A/B/B A/B/A/BA/B/B/AB/B/A/A

B/A/B/A B/A/A/B A/A/A/BB/A/A/AA/B/A/AA/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a standard therapy will include antiangiogenic therapy, chemotherapy, radiotherapy, immunotherapy, surgical therapy or gene therapy and may be employed in combination with the inhibitor of gene expression therapy, anticancer therapy, or both the inhibitor of gene expression therapy and the anti-cancer therapy, as described herein.

A. Antiangiogenic therapy

The skilled artisan will understand that additional antiangiogenic therapies may be used in combination or in conjunction with methods of the invention. For example additional antiangiogenic therapies may antagonize the VEGF and/or FGF signaling pathway. Thus, in some cases and additional therapy may comprise administration an antibody that binds to VEGF, a VEGF receptor, FGF or an FGF receptor. In certain specific aspects, methods and compositions of the invention may be used in conjunction with AVASTIN® (bevacizumab), LUCENTIS® (ranibizumab), MACUGEN® (pegaptanib sodium) or an anti-inflammatory drug. Thus, in certain specific cases there is provided a therapeutic composition comprising an anti-Olfml-3 composition and bevacizumab or pegaptanib sodium in a pharmaceutically acceptable carrier.

In still further aspects a gene that regulates angiogenesis may be delivered in conjunction with the methods of the invention. For example, in some aspects, a gene that regulates angiogenesis may be a tissue inhibitor of metalloproteinase, endostatin, angiostatin, endostatin XVIII, endostatin XV, kringle 1-5, PEX, the C-terminal hemopexin domain of matrix metalloproteinase-2, the kringle 5 domain of human plasminogen, a fusion protein of endostatin and angiostatin, a fusion protein of endostatin and the kringle 5 domain of human plasminogen, the monokine-induced by interferon-gamma (Mig), the interferon-alpha inducible protein 10 (IP10), a fusion protein of Mig and IP10, soluble FLT-1 (fins-like tyrosine kinase 1 receptor), and kinase insert domain receptor (KDR) gene. In certain specific aspects, such an angiogenic regulator gene may be delivered in a viral vector such as the lentiviral vectors described in U.S. Pat. No. 7,122,181, incorporated herein by reference.

B. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; carboplatin, procarbazine, plicomycin, gemcitabien, navelbine, farnesyl-protein tansferase inhibitors, transplatinum, and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

C. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

D. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gene silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

E. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that certain aspects of the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with certain aspects of the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increase of intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with certain aspects of the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### X. Kits and Diagnostics

In various aspects of the invention, a kit is envisioned containing therapeutic agents and/or other therapeutic and delivery agents. In some embodiments, the present invention contemplates a kit for preparing and/or administering a therapy of the invention. The kit may comprise one or more sealed vials containing any of the pharmaceutical compositions of the present invention. In some embodiments, the lipid is in one vial, and the nucleic acid component is in a separate vial. The kit may include, for example, at least one inhibitor of Olfml-3 expression, an Olfml-3 antibody, or an Olfml-3 protein or peptide, one or more lipid component, as well as reagents to prepare, formulate, and/or administer the components of the invention or perform one or more steps of the inventive methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

### XI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Differential Olfml-3 Gene Expression in tEnd.1V^{high} Angiogenic and tEnd.1V^{low} Resting Endothelial Cells Detected by Affimetrix Gene Chips

To identify novel genes involved in angiogenesis the inventors examined the gene expression profiles of tEnd.1V^{high} angiogenic and tEnd.1V^{low} resting endothelial cells by DNA microarray technique (GeneChip Mouse Genome 430 2.0 Array, Affimetrix). Normalization for each gene and comparative analysis between the expression profiles was carried out using GeneSpring GC 7.3 software. The data analysis resulted in 3500 differentially expressed genes in two cell lines, while >1700 genes showed two- or more-fold over-expression in tEnd.1V^{high} angiogenic cells.

To perform microarray data analysis and data mining, total RNA was extracted from mouse t.End.1V^{high} angiogenic and t.End.1V^{low} resting cells. The cultured cells were harvested and lysed using RNeasy Mini kit (Qiagen), according to the manufacturer's instructions. The purified RNA was quantified by a UV spectrophotometer, and RNA quality was evaluated by capillary electrophoresis on an Agilent 2100 Bioanalyser (Agilent Technologies). Total RNA was reverse transcribed using the cDNA synthesis kit (Roche). Labeled cDNA were hybridized to an Affimetrix Mouse Genome 430 2.0 Array GeneChip. Normalization for each gene and comparative analysis between expression profiles was carried out using GeneSpring GC 7.3 software. Comparative analysis was done with the data extracted from the NIH GEO Datasets database (available through world wide web at ncbi.nlm.nih.gov/entrez/query.fcgi?CMD=search&DB=gds).

In order to narrow down the number of target genes linked to angiogenesis, the microarray data were overlaid with the gene expression profiles provided by the National Institutes of Health Gene Expression Omnibus (GEO) public site (available through world wide web at ncbi.nlm.nih.gov/entrez/query.fcgi/). The GSE3601 microarray dataset contains angiogenesis-associated genes, differentially expressed by human umbilical cord vein endothelial cells (HUVEC) after lentiviral gene delivery of a vMIP-II protein (viral macrophage inflammatory protein II) with a potent proangiogenic activity (Cherqui et al., 2007).

Genes over-expressed at least 2× in tEnd.1V^{high} angiogenic cells (total of 612 genes) were compared to genes over-expressed at least 2× in activated HUVEC (total of 781 genes). Several genes that were identified by this comparative method are already associated with angiogenesis. However, using the same comparative method the inventors identified the mouse Olfml-3 gene with so far unknown function in angiogenesis. The Olfml-3 gene was 30-fold and 8.8-fold over-expressed in both mouse (this invention) and human angiogenic-associated microarray datasets (Cherqui et al., 2007).

In order to further validate the microarray result, the inventors used quantitative real-time RT-PCR (qPCR). Total RNA from tEnd.1V^{high} angiogenic and tEnd.1V^{low} resting cells were reverse-transcribed and subjected to qPCR.

More specifically, total RNA was extracted from tEnd.1V^{high} angiogenic cells 24 hours after nucleofection using RNeasy Mini kit (Qiagen). The isolated total RNA was quantified by a UV spectrophotometer and reverse transcribed using the cDNA synthesis kit (Roche), following manufacturer's instructions.

QPCR was performed according to the standard protocol used by in house facility (available through world wide web at frontiers-in-genetics.org/en/index.php?id=genomics). Primers used were the following: mouse Olfml3_RNAi-919 Forward:5′-GCTGTCTATGCCACTCGAGATG-′3 (SEQ ID NO:23); and Olfml3_RNAi-990 Reverse: 5′-TGTGTCAAGTGTCTGTGGGTCTAA-′3 (SEQ ID NO:24) as well as standard qPCR primers for three murine house keeping genes: β-actin, β-tubulin and EEF1A1. Reactions were performed in triplicate with the Power SYBR Green PCR kit and primer assay (Applied Biosystems, Inc) on a PCR system (Prism 7000; ABI). The results were quantified using the system software (SDS Prism 7000; ABI). Measurements of β-actin, β-tubulin and EEF1A1 house keeping genes were used for normalization of expression levels across samples.

The values for Olfml-3 were normalized with those of three house-keeping genes: β-actin, EEF1A1 and β-tubulin. The ratio of angiogenic and resting samples was calculated and shown as relative values (FIG. 1). Indeed, the 21-fold up-regulation of the Olfml-3 gene in angiogenic cells validated the selection of this gene as a novel angiogenesis associated target. The statistical analysis, using the Welch t-test, confirmed the significance of the data (p<0.00918).

### Example 2

### Vascular Specificity of Olfml-3 Gene Expression in Mouse Tissues

In situ mRNA hybridization on mouse tissue cryo-sections was performed with anti-sense riboprobes coding for the Olfml-3 gene, and PECAM-1 anti-sense was used as a vascular marker. Sense riboprobes served as a negative control in each in situ experiment. Using double labeling in situ mRNA method, the inventors observed strong co-localization of PECAM-1 and Olfml-3 expressing cells in several mouse tissues, thus confirming strong vascular specificity of the Olfml-3 gene. Namely, mOlfml-3 transcripts were detected in mouse endothelium of the highly vascularized organ heart (FIG. 2A, middle and right panel, arrows). More importantly, high level of Olfml-3 gene expression was found to co-localize with PECAM-1 expression in tumor vessels of mouse Lewis lung carcinoma (LLC1) (FIG. 2B, right panel), suggesting that Olfml-3 expression is strongly up-regulated in proliferative tissues.

This latter statement was confirmed by showing high levels of Olfml-3 gene expression in angiogenic vessels formed into a bFGF-loaded matrigel plug implanted in vivo (FIG. 2C, right panel). This is a standard method of choice for testing anti-angiogenesis strategies in vivo. This unique expression pattern suggests that high levels of the Olfml-3 gene are associated with vascular growth and remodeling in normal and pathological conditions.

To perform the double-labeling in situ mRNA hybridization to test vascular specificity of Olfml-3 gene expression, the digoxigenin (DIG)-labeled and fluorescein riboprobes were prepared after PCR amplification of mouse PECAM-1 gene with corresponding forward and reverse primers, latter containing the T7 polymerase binding site (underline) (mPECAM1-as-for1: ATG CTC CTG GCT CTG GGA CTC (SEQ ID NO:25) and mPECAM1-as-rev1: CTA ATA CGA CTC ACT ATA GGG TGC AGC TGG TCC CCT TCT ATG (SEQ ID NO:26); the mOlfm-3 gene with corresponding primers for sense and anti-sense riboprobes, respectively: mOlfml-3-s-for1: CTA ATA CGA CTC ACT ATA GGG AGT GCT CCT CTG CTG CTC CTC (SEQ ID NO:27); mOlfml-3-s-rev1: CGT GTC GTT CTG GGT GCC GTC (SEQ ID NO:28); mOlfml-3-as-for1: AGT GCT CCT CTG CTG CTC CTC (SEQ ID NO:29) and mOlfml-3-as-rev1: CTA ATA CGA CTC ACT ATA GGG CGT GTC GTT CTG GGT GCC GTC (SEQ ID NO:30).

Double labeling in situ hybridization was carried out on cryosections of mouse heart, LLC1 tumors or bFGF-treated matrigel plugs as following: cryosections were incubated in hybridization solution (50% formamide, 5×SSC, 0.1% Tween 20, 0.1% CHAPS, 1 Denhardt's solution, 0.01% heparine, 0.02% tRNA in DEPC H₂O) containing 1 to 2 μg/ml flourescein-labeled mouse PECAM-1 RNA probe and a DIG-labeled Olfml-3 RNA probe for 16 h at 55° C. For detection, samples were incubated with two antibodies simultaneously, the sheep anti-DIG Fab fragments coupled to alkaline phosphatase (1:2000, Roche) and the sheep anti-fluorescein Fab fragments coupled to horseradish peroxidase (1:100, Roche) during 1 h at RT. Unbound antibodies were removed using TNT buffer (150 mM NaCl, 100 mM Tris HCl, pH 7.5, 0.05% Tween-20) 3×5 min and incubated in biotinyl-tyramide mix diluted in the amplification buffer (1:50, PerkinElmer Life Sciences) for 30 min at RT, before being washed again in TNT buffer 3×5 min. Alexa-488-conjugated streptavidin antibody (1:100, Molecular Probes) was added in the amplification buffer and incubation was carried out at RT for 30 min. Cryosections were subsequently washed in TNT buffer 3×5 min and stained with Fast Red (DAKO Cytomation) for 30 min at RT in the dark. Staining was stopped by washing in TNT buffer 3×5 min at RT. Samples were stained with nuclear TO-PRO dye (Molecular probes; 1:2000), mounted in mowiol/DABCO (Sigma) mix and screened for fluorescent signals on a Zeiss LSM 510 confocal microscope.

For bFGF-treated matrigel assay in vivo, this Matrigel plug angiogenesis assay included implantation of Matrigel supplemented with the proangiogenic factor FGF-2 into mice. Implantation was performed via ventral and subcutanous injection of 400 microlitres matrigel loaded with 500 ng/ml b-FGF (FGF-2) per animal. To quantify matrigel plug vascularization, mice have been monitored once per week by intravenous injection of iodinated liposomes into the orbital plexus. These liposomes diffuse throughout the vascular system and can be detected by scanning with a micro computer tomograph Skyscan-1076, an X-ray imager. Mice were usually sacrificed at 21 days when the plugs reached acceptal vascularization levels. The plugs were then collected and analysed.

### Example 3

### Silencing of Olfml-3 Gene in tEnd.1V^{high} Angiogenic Cells

To further characterize the up-regulation of Olfml-3 in tEnd.1V^{high} angiogenic cells, the inventors used the small interfering RNAs (siRNAs) to knock-down Olfml-3 expression. The tEnd.1V^{high} angiogenic cells were transiently transfected using Nucleofector technology (Amaxa, Lonza Inc.). Three siRNAs were designed to target distinct regions of the Olfml-3 gene (Stealth™ Olfml-3 siRNA 1, 2 and 3, Invitrogen). The inventors obtained approximately 90% siRNA transfection efficiency, largely sufficient to detect functional effects of the targeted gene.

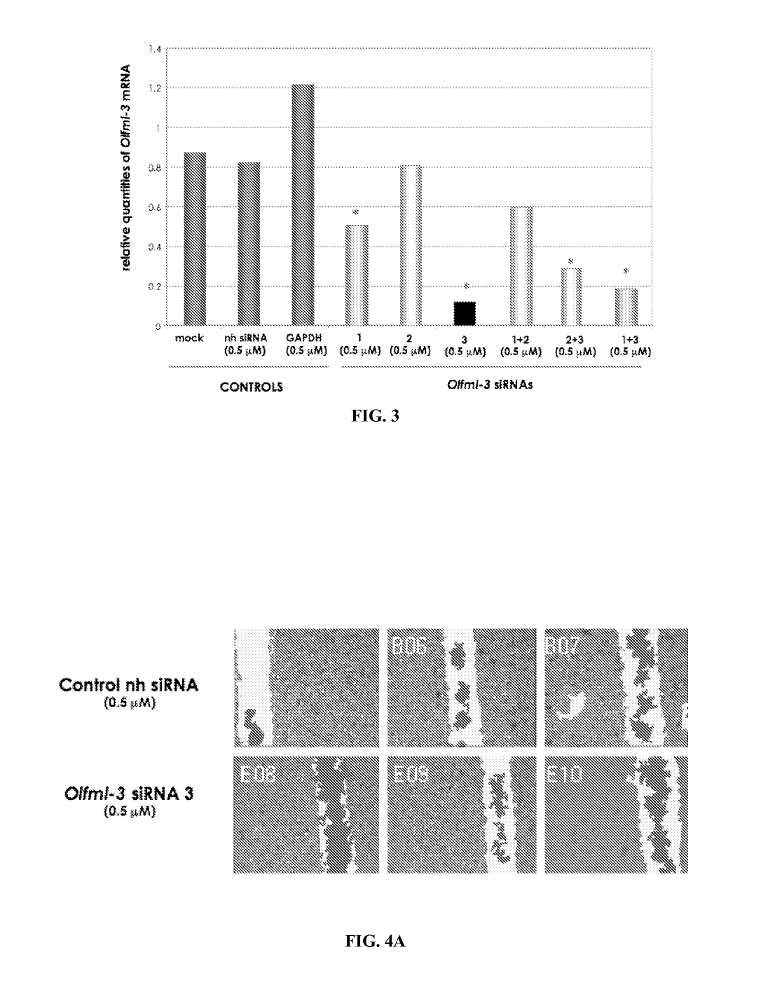

As a negative control (mock) the inventors used nucleofection without siRNA. The positive controls consisted of siRNAs for the GAPDH gene (GAPDH siRNA, Ambion) and the non-homologous sequence to mouse genes (nh siRNA, Ambion). The efficiency of silencing of Olfml-3 expression in the angiogenic cells was evidenced by qPCR 24 hours after nucleofection. As shown in FIG. 3, Olfml-3 siRNA 3 blocked the Olfml-3 gene expression by 95%. The Olfml-3 siRNA 1 appeared to be less efficient, reducing Olfml-3 gene expression only by 50% (FIG. 3) and Olfml-3 siRNA 2 failed to silence even when applied at higher concentrations. In order to observe possible additive effects of different siRNAs, three different combinations of the Olfml-3 siRNAs (siRNA 1+2, 2+3 and 1+3, 0.5 μM each) were transfected into angiogenic cells. The expression of the Olfml-3 gene was directly correlated with the presence of Olfml-3 siRNA 3, identifying it as the most potent silencer of the three. As expected, two control siRNAs failed to modify the Olfml-3 gene expression and the expression level was similar to the one observed for the mock controls (FIG. 3).

For cell cultures and transfection, the t.End.1V^{high} angiogenic and t.End.1V^{low} resting cells were cultured as previously described (Aurrand-Lions et al., 2004). They were use at low passages (up to third passage). Transient transfection of t.End.1V^{high} angiogenic cells were performed with a Nucleofector kit V (Amaxa) according to the manufacturer's instructions. For transfection, the following chemically modified duplex siRNAs were engaged: three siRNAs directed against mouse Olfml-3 gene (OLFML3MSS235376, OLFML3MSS235377, OLFML3MSS235378 here named as Olfml-3 siRNA 1, 2 and 3, respectively) (Stealth™ Select technology, Invitrogen), a siRNA against mouse GAPDH (Ambion) and a non-targeting negative control siRNA (nh siRNA) (Ambion). Single siRNAs or combinations of two different siRNAs were nucleofected at a concentration range of 0.4 to 0.6 μM. Transfected cells were engaged immediately after transfection in the experiments.

### Example 4

### Silencing of Olfml-3 Gene Leads to Modulation of Migratory Capacities of the t.End.1V^{high} Angiogenic Cells In Vitro

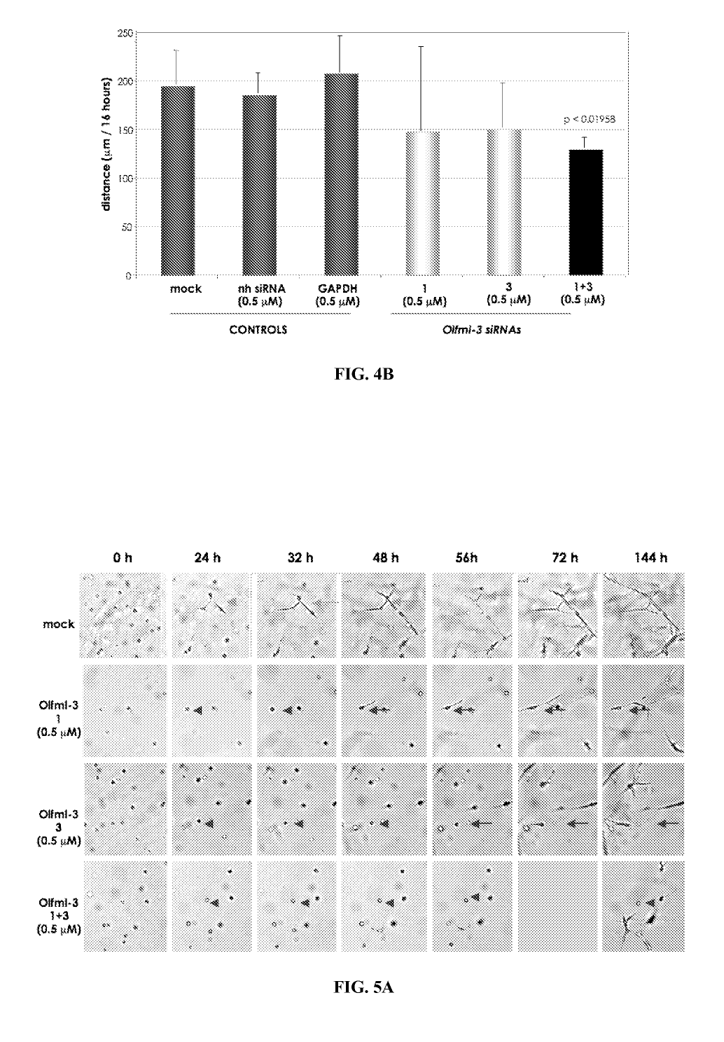

Since angiogenesis is dependent on cell migration the inventors further evaluated whether silencing of the Olfml-3 gene in t.End.1V^{high} angiogenic cells would affect their migration. This was tested by a wound-healing assay using matrigel-coated plates. The disruption of t.End.1V^{high} monolayers induced the cells at the edge of the wound to spread rapidly and migrate onto the matrigel. The leading front of the cell monolayer migrated homogenously as a unit during 16 hours (FIG. 4A). Photographs of the migrating cells were taken by the ImageExpress device at the beginning of the healing process and after 16 hours. This period was sufficient to obtain nearly closed wounds.

By using Metamorph software the inventors compared the distance of migration of control and Olfml-3-silenced t.End.1V^{high} angiogenic cells. The silenced cells migrated up to 40% less efficient when compared with control cells as shown in FIG. 4B. The highest reduction of cell migration was obtained with cells transfected with Olfml-1-3 siRNA 3 in which Olfml-3 silencing was the most efficient (FIG. 3). Cell migration was proportional to the level of silencing suggesting that Olfml-3 has a direct impact on cell migration. The evidence that silencing of the Olfml-3 gene can attenuate migration of t.End.1V^{high} angiogenic cells in vitro, suggests a functional importance of this molecule in angiogenesis.

In wound healing assay, 1.5×10⁴ t.End.1V^{high} angiogenic cells were seeded onto matrigel-coated 96-well plates and grown to confluence. Monolayers were wounded using a pipette tip and cell migration was monitored using an ImageXpress automated microscope equipped with 4× objective. The distance of migration was calculated using the Metamorph software. Wound healing assays for each sample were performed in triplicates and three independent experiments were carried out for each sample. Statistical analysis was preformed and standard deviation was calculated.

### Example 5

### Silencing of Olfml-3 Gene Attenuates the Initiation and the Final Steps of Sprout Formation In Vitro

In addition to the differences described above, the t.End.1V^{high} angiogenic cells form a capillary-like network of ramified cords in three-dimensional fibrin gels (Aurrand-Lions et al., 2004; Pepper et al., 1996). This so called sprouting assay represents a simple but powerful model for studying induction and/or inhibition of angiogenesis in vitro (Montesano et al., 1990; Pepper et al., 1996). Sprout formation starts with individual endothelial cells sending out spikes. These spikes initiate contacts with other cells in the vicinity; the cells then align and form capillary-like structures. The spikes of each cell can eventually initiate an alignment, which leads to a branched polygonal structure, resembling a capillary-like network.

As for tube formation assay in three-dimensional fibrin gels, fibrin gels were prepared as previously described (Pepper et al., 1996). The t.End.1V^{high} angiogenic cells were seeded in suspension into 100 μl of fibrin gels at 1.2×10⁴ cells per gel. Then 100 μl of DMEM containing 10% fetal calf serum and 200U of the proteinase inhibitor Trasylol (Aprotinin, Bayer) was added to each well above fibrin gels. During 6 days the cultures were photographed every 24 hours using an ImageXpress automated microscope. The number of sprouting cells per field was counted manually and statistical analysis was done on 3 to 10 fields per sample (standard deviation was calculated). Around 50-100 cells were analyzed per field. The total surface of vascular “skeleton” representing the capillary-like network was quantified using the Metamorph software.

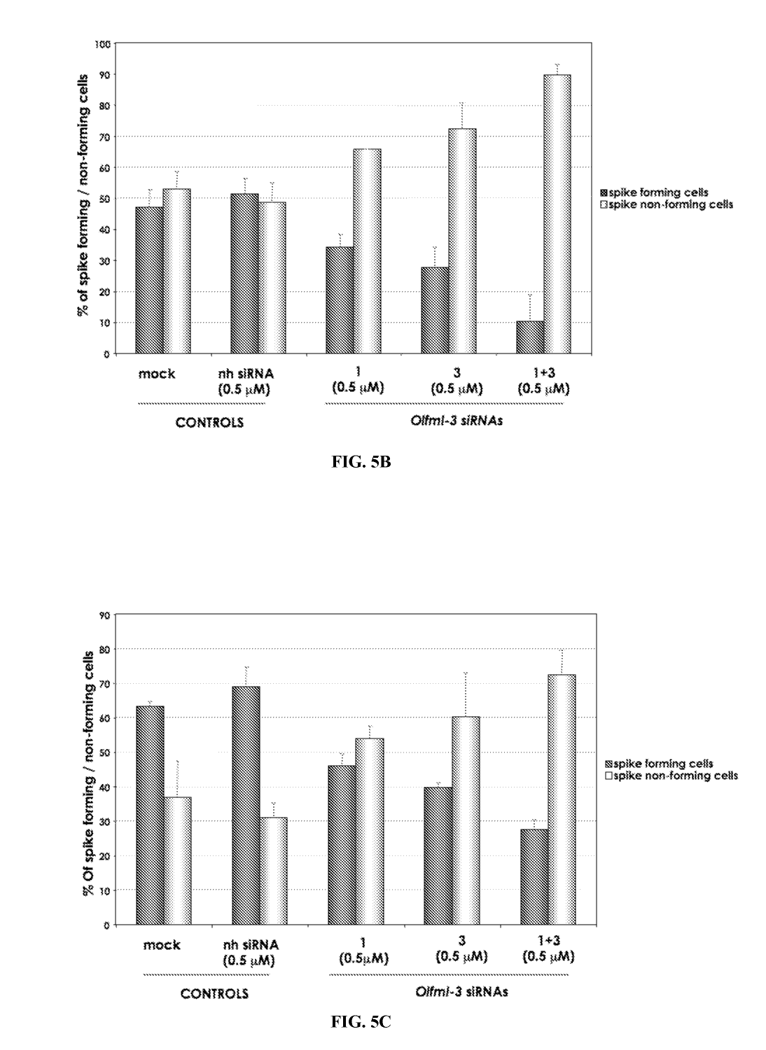

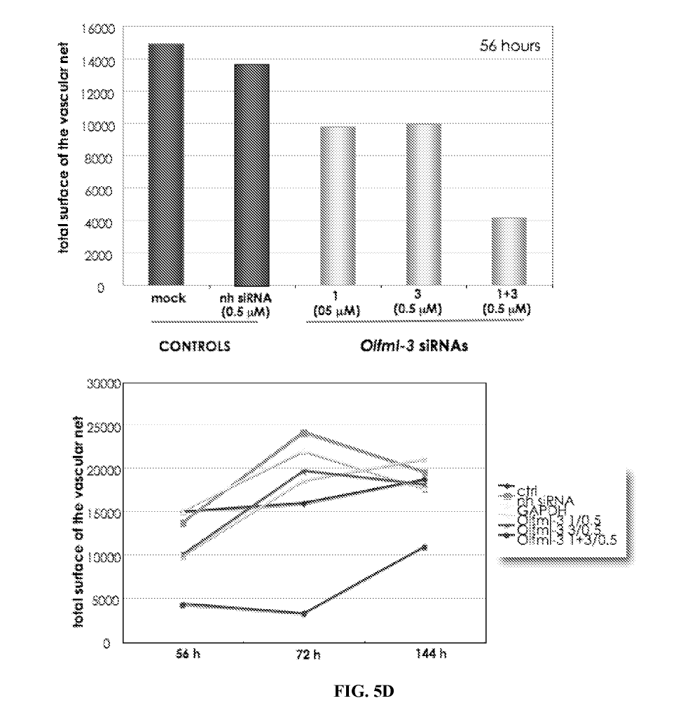

The inventors studied spike formation, the initial phase, and branching, the late phase of this angiogenic assay in vitro. Thus, the inventors silenced the Olfml-3 gene in the t.End.1V^{high} cells and used them in this assay. Photographs of the cultured endothelial cell were taken every 24 hours over six days (FIG. 5A). Around 50-100 cells were analyzed per field. About 50% of control, mock-transfected t.End.1V^{high} cells formed spikes within 24 hours, whereas silencing of Olfml-3 reduced the efficiency from 65% up to 10% only, depending on the siRNAs used (FIG. 5B). As a further control, transfection with nh or GAPDH siRNAs also did not show any difference. The early time point at 24 hours was important since it defines the potential degree of the future branching points. At 32 hours, about 65% of control, mock-transfected t.End.1V^{high} cells formed spikes, whereas silencing of Olfml-3 reduced the efficiency of spike formation up to 28% (FIG. 5C). At 56 hours the inventors measured the total surface of the vascular “skeleton” representing the capillary network. Development of this network decreased up to 20% when Olfml-3 was silenced when compared to the total vascular surface of control cells (FIG. 5D). This was not due to cell death, since the number of apoptotic cells was as low as 6.5-8.5% of total transfected cells even after 6 days (FIG. 5E). In conclusion, abrogation of Olfml-3 can attenuate the initiation and final steps of sprout formation in vitro, demonstrating the functional importance of Olfml-3 in modulating angiogenesis.

### Example 6

### Production of Recombinant Olfml-3 and Induction of Endothelial Sprouting

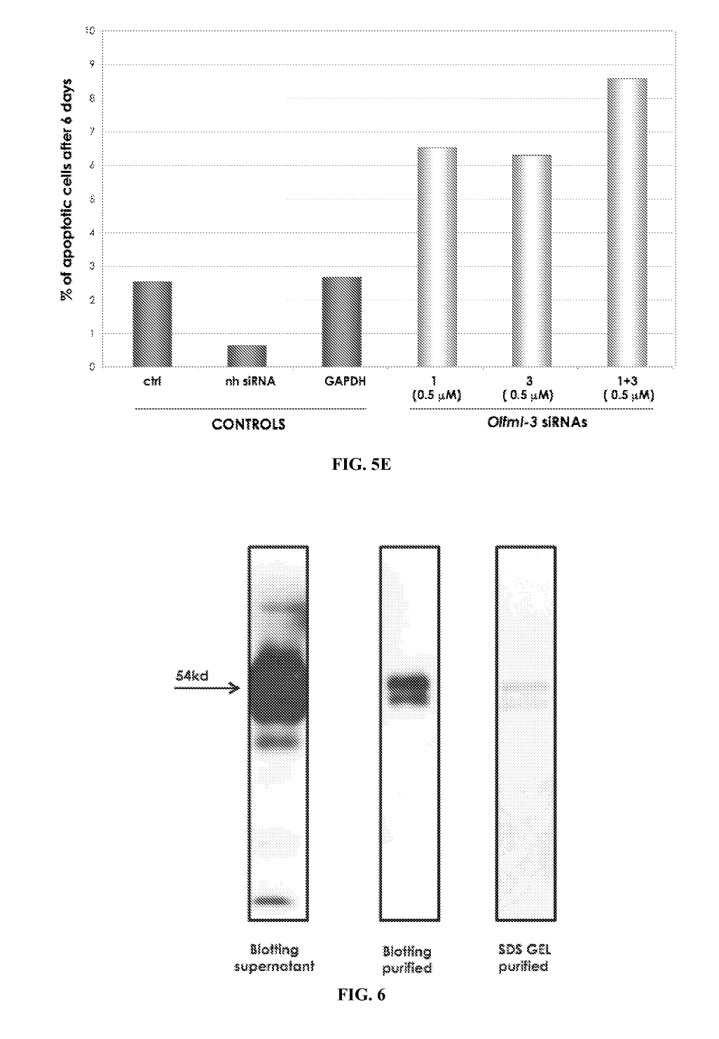

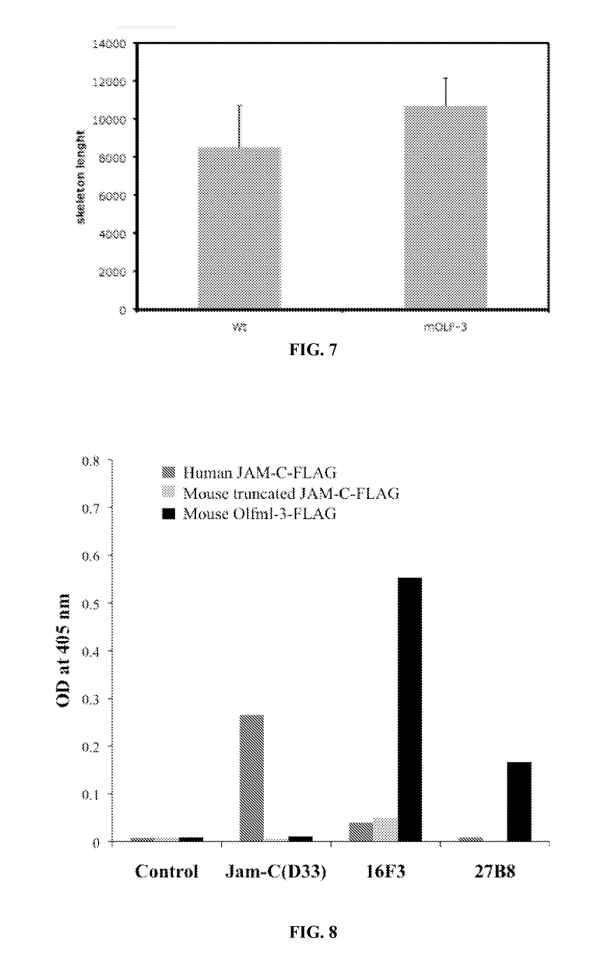

Full length mouse Olfml-3 gene was cloned as a FLAG tagged construct into the expression vector pcDNA3.3-TOPO. This vector was then transfected into MDCK epithelial cells and the producers selected by Neomycin resistance. The cell culture supernatant was then collected, the protein affinity purified and analyzed by Western blotting and SDS gels (FIG. 6). The protein appears as two bands with an expected average molecular weight of 54 kD suggesting that Olfml-3 was produced and secreted by the transfected cells. The two bands probably stem from different grades of glycolsylation. The Olfml-3 producing MDCK cells were then plated into a tissue well and overlaid by a fibrin gel containing t.End.1 endothelial cells. Length of the forming vascular skeleton was then determined using Metamorph software. Clearly, Olfml-3 secreted by MDCK cells increased vascular sprouting (FIG. 7). This is further evidence that Olfml-3 protein is needed for vascular angiogenesis.

Cloning strategy for the production of the recombinant Olfml-3 protein tagged with a FLAG sequence is as follows. The Olfml-3 full length cDNA was obtained by PCR, performed on MGC full length Olfml-3 clone in the pCMV-SPORT6 vector (Invitrogen, MGC cDNA clone: 7297, ID3485412). The Olfml-3 PCR fragment was inserted into the pcDNA 3.1 vector containing FLAG sequence (the pLig10-12, provided by C. Ody), where a FLAG sequence was inserted downstream to and in-frame with the Olfml-3 coding sequence. The Olfml-3-FLAG PCR fragment was than inserted into the pcDNA 3.3 TOPO TA vector (Invitrogen). This plasmid was multiplied in DH5a E. coli, purified by EndoFree Plasmid maxi preparation (Qiagen) and used for the production of the recombinant protein.

### Example 7

### Production of Monoclonal Antibodies

To analyse the expression of mouse Olfml-3 in more details, the inventors produced a panel of monoclonal antibodies against a recombinant Olfml-3 FLAG tagged protein (sOlfml-3-FLAG). The specificity of Olfml-3 monoclonal antibodies was addressed by performing ELISA assays using sOlfml-3-FLAG and the control proteins human JAM-C-FLAG recognized by the anti-JAM-C antibody D33, and truncated mouse JAM-C-FLAG not recognized by D33. Both monoclonal antibodies against Olfml-3 16F3 and 27B8 recognized specifically sOlfml-3-FLAG (FIG. 8). An anti-FLAG antibody recognized all three constructs (data not shown). These results confirmed the specificity of the selected monoclonal antibodies for mouse Olfml-3 protein.

The two antibodies against murine Olfml-3 were generated in the laboratory using standard techniques with recombinant soluble molecule as immunogen in rats (Aurrand-Lions et al., 1996). Briefly, 10 μg of purified recombinant Olfml-3-FLAG molecule (sOlfml-3-FLAG) mixed with adjuvant (Sigma) was used to immunize female Fischer rats. Two days after a final s.c. injection of 10 μg of sOlfml-3-FLAG, blasts form draining lymph nodes were fused to Sp2/0 cells, and hybridomas were selected in HAT-containing medium. Resistant clones were screened by ELISA for the production of monoclonal antibodies recognizing specifically sOlfml-3-FLAG. For this purpose, Maxisorb Immunoplates (Nunc) were coated overnight at 4° C. with M2 antibody diluted at 2 μg/ml in 150 mmNacl, 50 mm borate buffer, pH=9. Wells were washed, blocked for 1 h with serum-containing medium, and incubated for 1 h with supernatants of the sOlfml-3-FLAG transfected MDCK cells. After three washes with PBS plus 0.2% bovine serum albumin, hybridoma supernatants were added to the wells and incubated for 1 h at 4° C. After washing, bound antibodies were detected using mouse anti-rat peroxidase (Jackson Immunoresearch, Milan AG, La Roche, Switzerland), and ABTS (Sigma). Optical densities at 405 nm were read using a kinetic microplate reader and SoftMAXPro software (Molecular Devices Corp). Positive clones were subcloned twice, rescreened, and further tested. Two Olfml-3 antibodies: 16F3 and 27B8 are of IgG2a isotype subclass. Antibodies were purified on protein G-Sepharose columns (GE HealthCare) according to the manufacturer instructions. Specificity was assesed by ELISA using direct coating of different soluble molecules. The 16F3 and 27B8 monoclonal antibodies were used for in vivo tumor graft models.

### Example 8

### Anti-Olfml-3 Monoclonal Antibodies 16F3 and 27B8 Reduce Tumor Growth In Vivo

Given the finding that in vitro endothelial sprouting can be induced with Olfml-3 protein (FIG. 7), the inventors investigated whether the two monoclonal antibodies that were produced against mouse Olfml-3 affect tumor growth.

Eight- to 10-week-old female C56BL6/J mice were inoculated s.c. with 0.5×10⁶ murine Lewis lung carcinoma cells (LLC1; obtained from the European Collection of Cell Cultures, Salisbury, United Kingdom). Mice were then injected i.p. with the antibodies as follows: at day 1: 200 μg, at day 5: 200 μg and at day 8: 50 μg of monoclonal antibodies 16F3 and 27B8, isotype-matched control antibody mAb 64 (ctrl mAb 64), or PBS. When the control tumors (PBS-injected mice) had reached more than 0.5 cm, animals were sacrificed and tumors were excised and analysed. Tumor weight was measured. Volume was measured by using a caliper, applying the following formula for approximating the volume of an ellipsoid: Volume (mm³)=4/3π×(length/2)×(width/2)×(height/2).

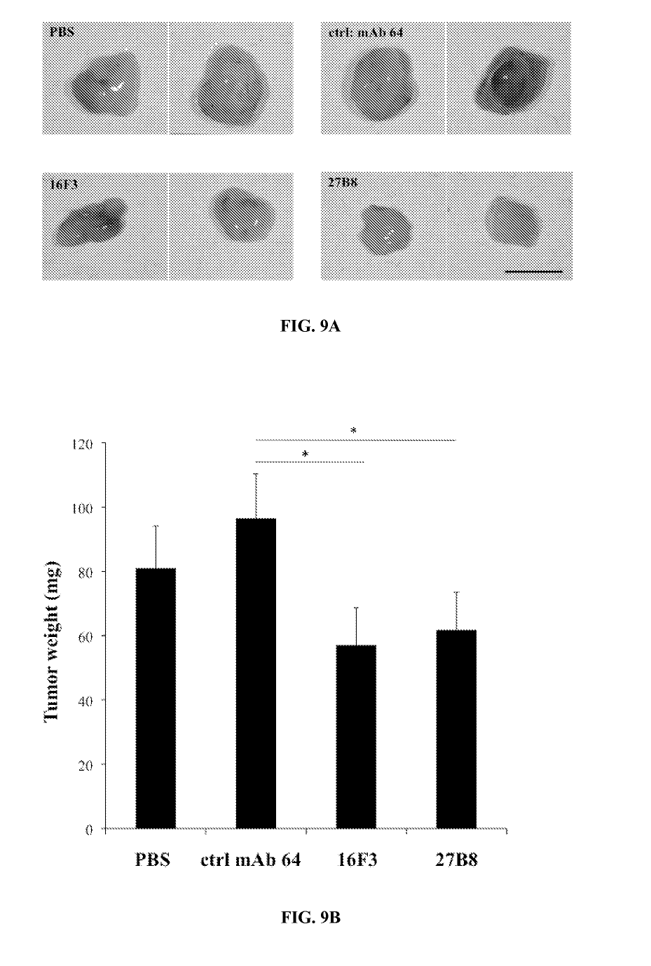

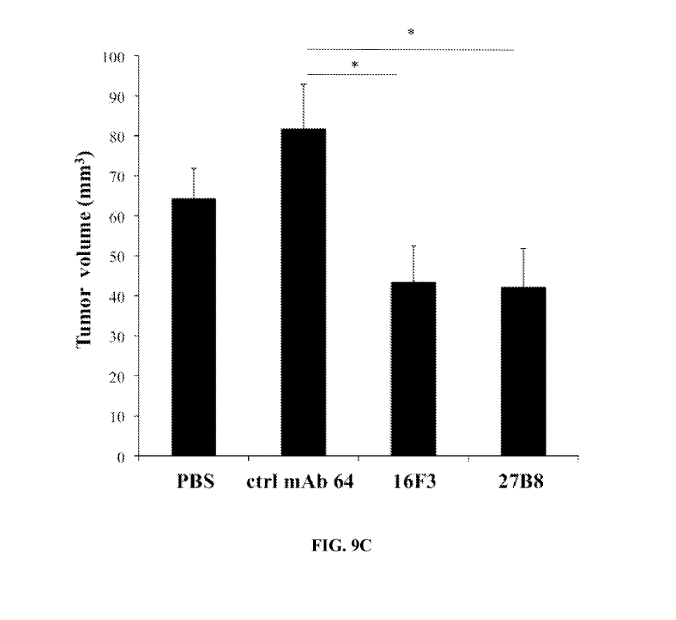

At day 9, animals were sacrificed and the tumors excised. The tumor volume and weight were significantly decreased in mice treated with either 16F3 or 27B8 anti-Olfml-3 antibodies compared to the isotype-matched control antibody (ctrl mAb 64) or PBS (FIGS. 9A-C). Because LLC1 tumor cells did not express Olfml-3 (data not shown) suggests, that the reduction in tumor growth was due to an effect of the two anti-Olfml-3 antibodies on tumor angiogenesis. This will serve as a proof-of-principle that anti-Olfml-3 antibodies can be used in anti-tumor therapy.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,469,797
U.S. Pat. No. 4,472,509
U.S. Pat. No. 4,606,855
U.S. Pat. No. 4,703,003
U.S. Pat. No. 4,742,159
U.S. Pat. No. 4,767,720
U.S. Pat. No. 4,816,567
U.S. Pat. No. 4,867,973
U.S. Pat. No. 4,870,287
U.S. Pat. No. 4,938,948
U.S. Pat. No. 4,946,778
U.S. Pat. No. 5,021,236
U.S. Pat. No. 5,091,513
U.S. Pat. No. 5,091,513
U.S. Pat. No. 5,164,296
U.S. Pat. No. 5,196,066
U.S. Pat. No. 5,223,409
U.S. Pat. No. 5,403,484
U.S. Pat. No. 5,420,253
U.S. Pat. No. 5,565,332
U.S. Pat. No. 5,571,698
U.S. Pat. No. 5,627,052
U.S. Pat. No. 5,656,434
U.S. Pat. No. 5,739,169
U.S. Pat. No. 5,760,395
U.S. Pat. No. 5,770,376
U.S. Pat. No. 5,789,208
U.S. Pat. No. 5,801,005
U.S. Pat. No. 5,821,337
U.S. Pat. No. 5,824,311
U.S. Pat. No. 5,830,880
U.S. Pat. No. 5,844,091
U.S. Pat. No. 5,846,945
U.S. Pat. No. 5,858,657
U.S. Pat. No. 5,861,155
U.S. Pat. No. 5,871,907
U.S. Pat. No. 5,969,108
U.S. Pat. No. 6,054,297
U.S. Pat. No. 6,165,464
U.S. Pat. No. 6,365,157
U.S. Pat. No. 6,406,867
U.S. Pat. No. 6,506,559
U.S. Pat. No. 6,573,099
U.S. Pat. No. 6,673,611
U.S. Pat. No. 6,709,659
U.S. Pat. No. 6,709,873
U.S. Pat. No. 6,753,407
U.S. Pat. No. 6,814,965
U.S. Pat. No. 6,849,259
U.S. Pat. No. 6,861,572
U.S. Pat. No. 6,875,434
U.S. Pat. No. 6,881,557
U.S. Pat. No. 6,891,024
U.S. Pat. No. 6,946,546
U.S. Patent Publn. 2002/0168707
U.S. Patent Publn. 2002/0172677
U.S. Patent Publn. 2003/0051263
U.S. Patent Publn. 2003/0055020
U.S. Patent Publn. 2003/0159161
U.S. Patent Publn. 2004/0064842
U.S. Patent Publn. 2004/0126828
U.S. Patent Publn. 2004/0265839
U.S. Patent Publn. 2004/0019001
U.S. Patent Publn. 2005/0214860
Ahmed et al., Invest. Ophthalmol. Vis. Sci., 45:3081-3090, 2004.
Aurrand-Lions et al., J. Pathol., 203:700-709, 2004.
Aurrand-Lions et al., Immunity, 5, 391-405, 1996.
Austin-Ward and Villaseca, Revista Medica de Chile, 126(7):838-845, 1998.
Bailey and Sullivan, Biochimica. Biophys. Acts., 239-252, 2000.
Bangham et al., J. Mol. Biol., 13(1):238-252; 253-259, 1965.
Barany and Merrifield, In: The Peptides, Gross and Meienhofer (Eds.), Academic Press, NY, 1-284, 1979.
Barbas et al., Proc. Natl. Acad. Sci., USA, 91:3809-3813, 1994.
Barembaum et al., Nat. Cell Biol., 2:219-225, 2000.
Bukowski et al., Clinical Cancer Res., 4(10):2337-2347, 1998.
Carmeliet, Nat. Med., 9:653-660, 2003.
Carmeliet, Nature, 438:932-936, 2005.
Cherqui et al., Mol. Ther., 15:1264-1272, 2007.
Christodoulides et al., Microbiology, 144(Pt 11):3027-3037, 1998.
Davidson et al., J. Immunother., 21(5):389-398, 1998.
Deamer and Uster, In: Liposome Preparation: Methods and Mechanisms, Ostro (Ed.), Liposomes, 1983.
Ferris et al., Arch. Ophthalmol., 102(11):1640-1642, 1984.
Fire et al., Nature, 391(6669):806-811, 1998.
Folkman, N. Engl. J. Med., 285:1182-1186, 1971.
Folkman, Nat. Med., 1:27-31, 1995.
Gariano and Gardner, Nature, 438:960-966, 2005.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gram et al., Proc. Natl. Acad. Sci. USA, 89:3576-3580, 1992.
Gregoriadis, In: Drug Carriers in Biology and Medicine, Gregoriadis (Ed.), 287-341, 1979.
Hanahan, Science, 277:48-50, 1997.
Hanibuchi et al., Int. J. Cancer, 78(4):480-485, 1998.
Harland, Cell, 134: 718-719, 2008.
Hellstrand et al., Acta Oncologica, 37(4):347-353, 1998.
Hu et al, Cancer Res., 56:3055-3061, 1996.
Hui and Hashimoto, Infection Immun., 66(11):5329-5336, 1998.
Ikeya et al., Int. J. Dev. Biol., 49:807-823, 2005.
Inomata et al., Cell, 134: 854-865, 2008.
Ju et al., Gene Ther., 7(19):1672-1679, 2000.
Kaneda et al., Science, 243:375-378, 1989.
Kato et al, J. Biol. Chem., 266:3361-3364, 1991.
Klein et al., Arch. Ophthalmol., 102:520-526, 1984.
Marks et al., Bio/Technol., 10:779-783, 1992.
Merrifield, Science, 232(4748):341-347, 1986.
Mitchell et al., Ann. NY Acad. Sci., 690:153-166, 1993.
Mitchell et al., J. Clin. Oncol., 8(5):856-869, 1990.
Montesano et al., Cell, 62:435-445, 1990.
Moreno and Bronner-Fraser, 2005
Morton et al., Arch. Surg., 127:392-399, 1992.
Nicolau et al., Methods Enzymol., 149:157-176, 1987.
Pepper et al., Enzyme Protein, 49:138-162, 1996.
Pietras et al., Oncogene, 17(17):2235-2249, 1998.
Qin et al., Proc. Natl. Acad. Sci. USA, 95(24):14411-14416, 1998.
Ravindranath and Morton, Intern. Rev. Immunol., 7: 303-329, 1991.
Rosenberg et al., Ann. Surg. 210(4):474-548, 1989.
Rosenberg et al., N. Engl. J. Med., 319:1676, 1988.
Rosenbauer et al., Blood, 103: 4294-4301, 2004.
Sakuragi et al., Mech Dev 123, 114-123, 2006.
Schier et al., Gene, 169(2):147-155, 1996.
Snyder et al., Biochemistry, 30:9143-9153, 1991.
Song et al., Stroke, 34:982-986, 2003.
Soutschek et al., Nature, 432(7014):173-178, 2004.
Stemmer, Nature 370:389-391, 1994.
Stewart and Young, In: Solid Phase Peptide Synthesis, 2d. ed., Pierce Chemical Co., 1984.
Szoka and Papahadjopoulos, Proc. Natl. Acad. Sci. USA, 75:4194 4198, 1978.
Tam et al., J. Am. Chem. Soc., 105:6442, 1983.
Tomarev and Nakaya, Mol. Neurobiol., 40: 122-138, 2009.
Tsuda et al., Neuron., 33:515-528, 2002.
Wadhwa et al., Curr. Opin. Mol. Ther., 6(4):367-372, 2004.
Wong et al., Gene, 10:87-94, 1980.
Yancopoulos et al., Nature, 407:242-248, 2000.
Yokoe and Anholt, Proc. Natl. Acad. Sci. USA, 90:4655-4659, 1993.
Zeng et al., FEBS Lett., 571:74-80, 2004.

1. An isolated nucleic acid molecule comprising a sequence that will hybridize with an Olfml-3 mRNA sequence selected from the group consisting of SEQ ID NOs: 1-7 and inhibit the expression of Olfml-3 in a cell.

2. The nucleic acid of claim 1, wherein the nucleic acid is an siRNA, a double stranded RNA, a short hairpin RNA, an antisense oligonucleotide, a ribozyme, a nucleic acid encoding thereof.

3. The nucleic acid of claim 2, wherein the nucleic acid is further defined as an siRNA or a nucleic acid encoding an siRNA.

4. The nucleic acid of claim 3, wherein the siRNA comprises SEQ ID NO: 8 or SEQ ID NO:10.

5. The nucleic acid of claim 4, wherein the siRNA comprises SEQ ID NO:10.

6. The nucleic acid of claim 4, wherein the siRNA comprises SEQ ID NO:8 and SEQ ID NO:10.

7. The nucleic acid of claim 4, wherein the siRNA comprises SEQ ID NO:9 and SEQ ID NO:10.

8. An antibody or a fragment thereof that binds to an Olfml-3 amino acid sequence selected from SEQ ID NOs: 11-17 and inhibits the activity of Olfml-3 in angiogenesis.

9. The antibody or fragment of claim 8, wherein the antibody is selected from the group consisting of a monoclonal antibody, a polyclonal antibody, a chimeric antibody, an affinity matured antibody, a humanized antibody, and a human antibody.

10. The antibody or fragment of claim 9, wherein the antibody is a monoclonal antibody.

11. The antibody or fragment of claim 9, wherein the antibody is a humanized antibody.

12. The antibody or fragment of claim 8, wherein the antibody fragment is a Fab, Fab′, Fab′-SH, F(ab′)₂, or scFv.

13. The antibody or fragment of claim 8, wherein the antibody or fragment is attached to an agent to be targeted to an Olfml-3-expressing cell.

14. The antibody or fragment of claim 13, wherein the agent is a cytotoxic agent, a cytokine, an anti-angiogenic agent, a chemotherapeutic agent, a diagnostic agent, an imaging agent, a radioisotope, a pro-apoptosis agent, an enzyme, a hormone, a growth factor, a peptide, a protein, an antibiotic, an antibody, a Fab fragment of an antibody, an imaging agent, an antigen, a survival factor, an anti-apoptotic agent, a hormone antagonist, a virus, a bacteriophage, a bacterium, a liposome, a microparticle, a magnetic bead, a microdevice, a cell, a nucleic acid or an expression vector.

15. A pharmaceutical composition comprising one or more said nucleic acids of claim 1 or said antibody or fragment of claim 8 in a pharmaceutically acceptable carrier.

16. The composition of claim 15, wherein the composition further comprises a lipid component.

17. The composition of claim 16, wherein the lipid component forms a liposome.

18. The composition of claim 16, wherein the lipid is 1,2-dioleoyl-sn-glycero-3-phosphatidylcholine (DOPC), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), palmitoyloeoyl phosphatidylcholine (“POPC”), lysophosphatidylcholine, dilinoleoylphosphatidylcholine distearoylphophatidylethanolamine (“DSPE”), dimyristoyl phosphatidylethanolamine (“DMPE”), dipalmitoyl phosphatidylethanolamine (“DPPE”), palmitoyloeoyl phosphatidylethanolamine (“POPE”), lysophosphatidylethanolamine, phosphatidylserine, phosphatidylglycerol, dimyristoyl phosphatidylserine (“DMPS”), dipalmitoyl phosphatidylserine (“DPPS”), brain phosphatidylserine (“BPS”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), or dioleoylphosphatidylglycerol (“DOPG”).

19. The composition of claim 15, wherein the composition further comprises cholesterol or polyethyleneglycol (PEG).

20. A method of treating an angiogenesis-related condition in a subject comprising administering to the subject an amount of a composition in accordance with claim 15 that is effective to treat the angiogenesis-related condition.

21. The method of claim 20, wherein the composition comprises the. nucleic acids.

22. The method of claim 20, wherein the composition comprises. the antibody or fragment thereof.

23. The method of claim 20, wherein the subject is a human subject.

24. The method of claim 20, wherein the angiogenesis-related condition comprises cancer.

25. The method of claim 24, wherein the cancer is breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

26. The method of claim 20, wherein the angiogenesis-related conditions is ocular neovascularization, arterio-venous malformations, coronary restenosis, peripheral vessel restenosis, glomerulonephritis, rheumatoid arthritis, pancreatitis, bowl diseases, ischemic cardiovascular pathologies, or chronic inflammatory diseases.

27. A pharmaceutical composition for inducing angiogenesis in a subject, comprising:
(a) an isolated Olfml-3 protein or peptide comprising at least 10 amino acids having at least 95% identity to an amino acid sequence selected from the group consisting of SEQ ID NO:11-17; and
(b) a pharmaceutically acceptable carrier.

28. The composition of claim 27, wherein the composition further comprises a lipid component.

29. The composition of claim 28, wherein the lipid component forms a liposome.

30. The composition of claim 28, wherein the lipid is 1,2-dioleoyl-sn-glycero-3-phosphatidylcholine (DOPC), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), palmitoyloeoyl phosphatidylcholine (“POPC”), lysophosphatidylcholine, dilinoleoylphosphatidylcholine distearoylphophatidylethanolamine (“DSPE”), dimyristoyl phosphatidylethanolamine (“DMPE”), dipalmitoyl phosphatidylethanolamine (“DPPE”), palmitoyloeoyl phosphatidylethanolamine (“POPE”), lysophosphatidylethanolamine, phosphatidylserine, phosphatidylglycerol, dimyristoyl phosphatidylserine (“DMPS”), dipalmitoyl phosphatidylserine (“DPPS”), brain phosphatidylserine (“BPS”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), or dioleoylphosphatidylglycerol (“DOPG”).

31. The composition of claim 27, wherein the composition further comprises cholesterol or polyethyleneglycol (PEG).

32. A method for treating an angiogenesis-related condition comprising administering to a subject in need of angiogenesis an amount of a composition in accordance with claim 27 that is effective to induce angiogenesis.

33. The method of claim 32, wherein the subject is a human subject.

34. The method of claim 32, wherein the angiogenesis-related condition is transplantation, cardiovascular diseases, aneurisms or wound healing.

35. The method of claim 34, wherein the angiogenesis-related condition is wound healing.
